(19) United States
(12) Patent Application Publication
(10) Pub. No.: US 2024/0358652 A1
(43) Pub. Date: Oct. 31, 2024

(54) LIPID NANOPARTICLE FORMULATIONS

(71) Applicant: Monash University, Clayton, Victoria (AU)

(72) Inventors: Hareth Basim Ali AL-WASSITI, Clayton, Victoria (AU); Colin William POUTON, Clayton, Victoria (AU); Kha Tu Joan HO, Clayton, Victoria (AU)

(73) Assignee: Monash University, Clayton, Victoria (AU)

(21) Appl. No.: 18/291,808

(22) PCT Filed: Aug. 17, 2022
(86) PCT No.: PCT/AU2022/050913

§ 371 Date: Jan. 24, 2024

(30) Foreign Application Priority Data
Aug. 17, 2021 (AU) 2021902567

(51) Int. Cl. A61K 9/51; A61K 31/713; A61K 39/00; A61K 48/00; A61P 37/08

(52) U.S. Cl. CPC A61K 9/5146; A61K 9/5123; A61K 31/713; A61K 39/0005; A61K 48/0041; A61P 37/08; A61K 2039/53; A61K 2039/55555; A61K 2039/572

(57) ABSTRACT

The present invention is directed to lipid nanoparticles, formulations containing lipid nanoparticles and methods of treating diseases or conditions with said lipid nanoparticles and formulations thereof. A lipid nanoparticle comprising (a) an active agent; (b) a cationic and/or ionisable lipid comprising from about 40 mol % to about 60 mol % of the total lipid present in the nanoparticle; (c) a phospholipid comprising from about 5 mol % to about 20 mol % of the total lipid present in the nanoparticle; (d) a structural lipid comprising from about 30 mol % to about 50 mol % of the total lipid present in the nanoparticle; (e) a PEGylated lipid comprising from about 0.05 mol % to less than 0.5 mol % of the total lipid present in the nanoparticle.

### FIELD OF THE INVENTION

The present invention is directed to lipid nanoparticles, formulations containing lipid nanoparticles and methods of treating diseases or conditions with said lipid nanoparticles and formulations thereof.

### RELATED APPLICATION

This application claims priority from Australian provisional application AU 2021902567, the entire contents of which are hereby incorporated by reference.

### BACKGROUND OF THE INVENTION

The effective targeted delivery of biologically active substances such as small molecule drugs, proteins, and nucleic acids represents a continuing medical challenge. In particular, the delivery of nucleic acids to cells is made difficult by the relative instability and low cell permeability of such species. Thus, there exists a need to develop methods and compositions to facilitate the delivery of therapeutic and/or prophylactics such as nucleic acids to cells.

Lipid-containing nanoparticle compositions, liposomes, and lipoplexes have proven effective as transport vehicles into cells and/or intracellular compartments for biologically active substances such as small molecule drugs, proteins, and nucleic acids. Such compositions generally include one or more “cationic” and/or amino (ionizable) lipids, phospholipids including polyunsaturated lipids, structural lipids (e.g., sterols), and/or lipids containing polyethylene glycol (PEG lipids). Cationic and/or ionizable lipids include, for example, amine-containing lipids that can be readily protonated.

Efforts have been directed toward improving delivery efficacy of lipid nanoparticle formulations. Many such efforts have been aimed toward developing more appropriate cationic lipids. Despites these efforts, improvements in terms of increased efficacy are still needed, especially for lipid nanoparticle based drug delivery systems intended for therapeutic uses.

Accordingly, there is a need for new and/or improved lipid nanoparticles with increased efficacy, and in particular for altered organ distribution.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a lipid nanoparticle comprising:
(a) an active agent;
(b) a cationic and/or ionisable lipid comprising from about 40 mol % to about 60 mol % of the total lipid present in the nanoparticle;
(c) a phospholipid comprising from about 5 mol % to about 20 mol % of the total lipid present in the nanoparticle;
(d) a structural lipid comprising from about 30 mol % to about 50 mol % of the total lipid present in the nanoparticle;
(e) a PEGylated lipid comprising from about 0.05 mol % to less than 0.5 mol % of the total lipid present in the nanoparticle.

In preferred embodiments, the active agent or therapeutic agent is fully encapsulated within the lipid portion of the lipid particle such that the active agent or therapeutic agent in the lipid particle is resistant in aqueous solution to enzymatic degradation, e.g., by a nuclease or protease. In other preferred embodiments, the lipid particles are substantially non-toxic to mammals such as humans.

In one embodiment, the lipid nanoparticle does not comprise a targeting ligand that specifically binds to a molecule on the surface of the target cell. Alternatively, the lipid nanoparticle is targeting ligand free.

In another aspect, the present invention provides a pharmaceutical composition comprising a lipid nanoparticle of the invention and a pharmaceutically acceptable carrier, diluent or excipient.

In another aspect, the present invention provides a method for introducing an active agent (e.g. nucleic acid) into a cell, preferably the cell is present in vivo, the method comprising contacting the cell with a lipid nanoparticle of the invention, thereby introducing the active agent (e.g. nucleic acid) into the cell.

In another aspect, the present invention provides a method for the in vivo delivery of an active agent, the method comprising administering a lipid nanoparticle of the invention to a subject in need thereof, thereby delivering an active agent to the subject.

In another aspect, the present invention provides a method for treating or preventing a disease or condition in a subject in need thereof, the method comprising administering to the subject a lipid nanoparticle or pharmaceutical composition of the invention, thereby treating or preventing a disease or condition in a subject in need thereof.

In another aspect, the present invention provides a lipid nanoparticle or pharmaceutical composition of the invention in the manufacture of a medicament for treating or preventing a disease or condition in a subject in need thereof.

In another aspect, the present invention provides lipid nanoparticle or pharmaceutical composition of the invention for use in the treatment or prevention of a disease or condition in a subject in need thereof.

In another aspect, the present invention provides a method of producing a polypeptide of interest in a cell, preferably a mammalian cell, the method comprising contacting the cell with a lipid nanoparticle of the invention, wherein the active agent is an mRNA encoding the polypeptide of interest, wherein the mRNA is capable of being translated in the cell to produce the polypeptide of interest.

In another aspect, the present invention provides a method of delivering an mRNA into a cell, preferably a mammalian cell, the method comprising administering to a subject a lipid nanoparticle of the invention, wherein the active agent is an mRNA, thereby delivering an mRNA into a cell.

In any aspect or embodiment, the cell is a mammalian cell.

In any aspect or embodiment, the cell is a cell located in the spleen. The cell located in the spleen may be a cell of the spleen or a cell originating in another part of the subject that is trafficked to the spleen.

In any embodiment, the lipid nanoparticle preferentially targets the spleen in comparison to the liver. In one embodiment, the lipid nanoparticle has a spleen/liver targeting ratio of greater than 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, or 14.5. In one embodiment, the lipid nanoparticle has a spleen/liver targeting ratio of greater than or equal to about 8, or about 14, or any other value described herein including the Examples and Figures. Typically, the spleen/liver targeting ratio is determined using an assay as described herein, including the Examples, such as a nanoluciferase assay.

In another aspect, the present invention provides a method for the delivery of an mRNA to a target tissue, the method comprising administering to a subject a lipid nanoparticle of the invention, wherein the active agent is an mRNA, thereby delivering the mRNA to a target tissue.

Preferably, the target tissue is a mammalian spleen.

In another aspect, the present invention provides a process for producing a lipid nanoparticle of the invention, the process comprising;
mixing a solution of cationic and/or ionisable lipid, phospholipid, structural lipid and PEGylated lipid to yield the desired molar ratios,
thereby producing a lipid nanoparticle.

Preferably, the process further includes adding the active agent to the step of mixing the lipids. In one embodiment, the lipid nanoparticles are formed where the lipid components to the active agent are at wt:wt ratios between about 5:1 and about 50:1. In one embodiment, the final lipid concentration in the solution is between about 5.5 mM and about 50 mM, preferably diluted with ethanol.

The mixing a solution of cationic and/or ionisable lipid, phospholipid, structural lipid and PEGylated lipid to yield the desired molar ratios may form a lipid solution. In one embodiment, the lipid solution is rapidly injected using a microfluidics system, such as a Nano-Assembler microfluidic based system, at flow rates between about 0.5 ml/min and about 8 ml/min into the a solution containing the active agent thereby producing a suspension with a water to ethanol ratio between about 1:1 and about 4:1, preferably 3:1.

Typically, the NP ratio (nitrogen to phosphate) is maintained between 4-7.

In one embodiment, the process of producing the lipid nanoparticles of the invention includes one or more of, or all of, the steps described in the Examples herein, e.g. Example 1.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Illustration of lipid nanoparticle generation using the NanoAssembler.

FIG. 2. Particle size and polydispersity index of MIPS-LNP. (A) Initial particle size and polydispersity index (PDI) of typical MIPS-LNP (n=3), which shows the particle falls within a range of both size and dispersity that is pharmaceutically acceptable. (B) Size distribution of MIPS-LNPs versus conventional LNPs. (C) Stability of particles produced using DMG-PEG at Day 0, Day 7 and 6 months.

FIG. 3. Spleen delivery preference of MIPS-LNP using DODAP lipid. The Pie chart shows proportional gene expression in tissues outside of the muscle after IM injection. Outside the muscle, MIPS-LNP (A) delivers >75% of the dose to the spleen (pink) with active delivery to both lymph nodes (DLN and nonDLN), while most of the dose of conventional LNP outside the muscle tissue (B) enters the liver after IM injection, which mirrors to some extent to the distribution of particles after IV injection. Key to tissue: non-DLN—non-draining lymph nodes, DLN—draining lymph nodes, SP—spleen, KD—kidney, LG—lung, LV—liver.

FIG. 4. MIPS-LNPs enhance gene delivery and expression within the spleen compared to conventional LNPs. (A) Intramuscular injection of MIPS-LNP formulation in mice results in ˜100× higher gene expression levels in spleen compared to conventional LNP (highlighted box). (n=3 or 4). Key to tissue: non-DLN—non-draining lymph nodes, DLN—draining lymph nodes, SP—spleen, KD—kidney, LG—lung, LV—liver, QM—quadriceps muscle. (B) Intravenous injection of MIPS-LNP formulation (using DODAP) in mice results in ˜500× higher gene expression levels in spleen than conventional LNP (highlighted box). (n=2 or 3). LN1 and LN2—sample lymph nodes. (C) Analysis of sorted splenocytes following intravenous injection of MIPS-LNP display enhanced gene expression levels compared with conventional LNPs 10 μg of DODAP formulated nanoluciferase mRNA was injected in all experiments.

FIG. 5. Illustration of ovalbumin-targeted vaccine mouse model protocol. Analysis was performed using flow-cytometer and FloJo software.

FIG. 6. MIPS-LNPs induce enhanced target-specific cytotoxic T cell-killing against ovalbumin epitopes (CD8+ cell response). In vivo killing of ovalbumin-pulsed target cells by cytotoxic T cells after vaccination with mRNA encoding ovalbumin. Data presented as percentage killing compared to unpulsed cells. A minimum of n=3 per treatment was used. Data shows at 10 μg dose, MIPS formulation induces full cell killing activity while standard (Conventional) LNPs do not.

FIG. 7. Particle size of LNPs before and after freeze-thaw cycles in various buffers with or without sucrose (Suc). Different Tris-buffers compositions were used. Sucrose was also used as protectant. No major difference between the buffers but Sucrose was important for freeze-thawing stability of MIPS formulation.

FIG. 8. Nanoluciferase levels expressed as relative light units per unit mass of tissue following intravenous (A) or intramuscular (quadriceps) (B) delivery of nanoluciferase mRNA in LNP formulations containing either DSPE-PEG or DMG-PEG at either 0.15 mole % or 1.5 mole %. The LNP formulations contained DLin-MC3-DMA (50 mole %), distearoylphosphatidylcholine (DSPC) (10 mole %), PEGylated lipid (0.15 mole % or 1.5 mole %), cholesterol (remainder of the lipid content—i.e 39.85 mole % or 38.5 mole % respectively). (A) ANOVA with post hoc pairwise compassion. * p<0.05, ** p<0.01; (B) ANOVA with post hoc pairwise compassion. * p<0.05, ** p<0.01, dotted line shows data analysed by t-test * p<0.05. Key to tissues: LN1 and LN2—sample lymph nodes, DLN—draining lymph node after IM injection, nDLA—non draining lymph node (alternate limb), QM—quadriceps muscle.

FIG. 9. Statistical comparison of intramuscular data (shown also in FIG. 8) for LNP formulations containing DMG-PEG. DLinMC3 DMA was used in MIPS formulation in this experiment.

FIG. 10. Tissue targeting to spleen after intramuscular injection is indicated by comparing the nanoluciferase activity in spleen and liver as a ratio. (A) Both DMG-PEG and DSPE-PEG formulations containing 0.15 mole % PEGylated lipid targeted the spleen after IM injection. (B) Targeting to the spleen versus liver is more pronounced at low concentrations of DMG-PEG

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth. For purposes of interpreting this specification, terms used in the singular will also include the plural and vice versa.

The present invention is based on the surprising development of lipid nanoparticles that result in increased transfection of cells in the spleen. This is surprising as the lipid nanoparticles do not contain a targeting ligand that specifically binds to a molecule on the surface of a cell in the spleen (e.g. a splenocyte). Further, the increase in infection of cells is specific to the spleen as, compared to convention lipid nanoparticles, the lipid nanoparticles of the invention do not significantly increase transfection in one or more other sites in the body (e.g. heart, liver, kidney, lung, blood, and/or lymph nodes). This enhanced transfection of the spleen is independent of administration route as it is observed when the lipid nanoparticle of the invention are administered intravenously or intramuscularly. Further, the advantages provided by the lipid nanoparticles of the invention only occur when the PEG-lipid content of the lipid nanoparticles are within a narrow mol % range of the total lipid of the nanoparticle.

This lower and narrower mol % range of PEG-lipid results in a nanoparticle that is equal to or larger than 100 nm in diameter, which is larger than the size range of 70-100 nm diameter of current lipid nanoparticles. Typically, the lipid nanoparticles of the invention are larger than 125 nm in diameter, such as 140-160 nm.

Another property of the lipid nanoparticle of the invention is the unusually highly negative zeta potential. Nanoparticles with relatively low charges, positive or negative, are generally desirable, as more highly charged species may interact undesirably with cells, tissues, and other elements in the body. However, the lipid nanoparticles of the present invention have a high negative zeta potential and surprisingly still display increased passive targeting in the spleen.

Finally, the lipid nanoparticles of the invention are stable for an extended period of time, for example at least 5 months at 4° C.

Without being bound by any theory or mode of action, it is believed that the increased particle size enhances interaction and update by phagocytic cells, and along with the highly negative zeta potential, drives spleen delivery and transfection of antigen presenting cells.

While the lipid nanoparticles have many applications, such as those described herein, the enhanced transfection of cells of the spleen provides a particularly beneficial delivery vehicle for antigenic or immunogenic molecules (or molecules that induce the cell to produce antigenic or immunogenic molecules) such as those found in vaccines. Splenocytes targeting is attractive for many applications such as expression of proteins for immunocheckpoint inhibition and other applications, through the targeted mRNA delivery by MIPS formulation, that induce antigen-specific tolerance, induction general tolerance and applications in countering autoimmune diseases, reducing inflammation driven by splenocytes or reducing allergy and anaphylactic reactions through means of mRNA delivery, siRNA delivery, DNA or any other nucleic acid or mRNA delivery with other molecules (Such as small drugs incorporated with MIPS LNPs).

### Definitions

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean +/−10% of the recited value. For instance, a nanoparticle composition including a lipid component having about 40% of a given compound may include 30-50% of the compound.

### Nanoparticles

The mean size of a nanoparticle of the invention may be greater than about 100 nm e.g., measured by dynamic light scattering (DLS). For example, the mean size may be greater than, or great than about, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, or 500 nm.

In some embodiments, the mean size of a nanoparticle of the invention may be from about 75 nm to about 500 nm, from about 80 nm to about 500 nm, from about 90 nm to about 500 nm, from about 100 nm to about 500 nm, from about 110 nm to about 500 nm, from about 120 nm to about 500 nm, from about 130 nm to about 500 nm, from about 140 nm to about 500 nm, from about 150 nm to about 500 nm, from about 160 nm to about 500 nm, from about 170 nm to about 500 nm, from about 180 nm to about 500 nm, from about 190 nm to about 500 nm, from about 200 nm to about 500 nm, from about 210 nm to about 500 nm, from about 220 nm to about 500 nm, from about 230 nm to about 500 nm, from about 240 nm to about 500 nm, from about 250 nm to about 500 nm, from about 260 nm to about 500 nm, from about 270 nm to about 500 nm, from about 280 nm to about 500 nm, from about 300 nm to about 500 nm, from about 310 nm to about 500 nm, from about 320 nm to about 500 nm, from about 330 nm to about 500 nm, from about 340 nm to about 500 nm, from about 350 nm to about 500 nm, from about 360 nm to about 500 nm, from about 370 nm to about 500 nm, from about 380 nm to about 500 nm, from about 390 nm to about 500 nm, from about 400 nm to about 500 nm, from about 410 nm to about 500 nm, from about 420 nm to about 500 nm, from about 430 nm to about 500 nm, from about 440 nm to about 500 nm, from about 450 nm to about 500 nm, from about 460 nm to about 500 nm, from about 470 nm to about 500 nm, from about 480 nm to about 500 nm, or from about 490 nm to about 500 nm.

In some embodiments, the mean size of a nanoparticle of the invention may be from about 100 nm to about 490 nm, from about 100 nm to about 480 nm, from about 100 nm to about 470 nm, from about 100 nm to about 460 nm, from about 100 nm to about 450 nm, from about 100 nm to about 440 nm, from about 100 nm to about 430 nm, from about 100 nm to about 420 nm, from about 100 nm to about 430 nm, from about 100 nm to about 420 nm, from about 100 nm to about 410 nm, from about 100 nm to about 400 nm, from about 100 nm to about 390 nm, from about 100 nm to about 380 nm, from about 100 nm to about 370 nm, from about 100 nm to about 360 nm, from about 100 nm to about 350 nm, from about 1000 nm to about 340 nm, from about 100 nm to about 330 nm, from about 100 nm to about 320 nm, from about 100 nm to about 310 nm, from about 100 nm to about 300 nm, from about 100 nm to about 290 nm, from about 100 nm to about 280 nm, from about 100 nm to about 270 nm, from about 100 nm to about 260 nm, from about 100 nm to about 250 nm, from about 100 nm to about 240 nm, from about 100 nm to about 230 nm, from about 100 nm to about 220 nm, from about 100 nm to about 210 nm, from about 100 nm to about 200 nm, from about 100 nm to about 190 nm, from about 100 nm to about 180 nm, from about 100 nm to about 170 nm, from about 100 nm to about 160 nm, from about 100 nm to about 150 nm, from about 100 nm to about 140 nm, from about 100 nm to about 130 nm, from about 100 nm to about 120 nm, or from about 100 nm to about 110 nm.

A nanoparticle may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

As used herein, the “zeta potential” is the electrokinetic potential of a lipid, e.g., in a nanoparticle.

The zeta potential of a nanoparticle may be used to indicate the electrokinetic potential of the particle. For example, the zeta potential may describe the surface charge of a nanoparticle. Nanoparticles with relatively low charges, positive or negative, are generally desirable, as more highly charged species may interact undesirably with cells, tissues, and other elements in the body. However, present lipid nanoparticles have unusually highly negative zeta potential. In some embodiments, the zeta potential of a nanoparticle may be from about −50 mV to about +10 mV, preferably about −10 mV to about +5 mV. In some embodiments, the zeta potential of a nanoparticle may be from −50 mV to +10 mV, preferably −10 mV to +5 mV. Further, in some embodiments, the zeta potential of a nanoparticle may be from about −20 mV to about −5 mV, from about −15 mV to about −5 mV, from about −10 mV to about −5 mV, from about −20 mV to about −10 mV. Further, in some embodiments, the zeta potential of a nanoparticle may be from −20 mV to −5 mV, from −15 mV to −5 mV, from −10 mV to −5 mV, from −20 mV to −10 mV. Further, in some embodiments, the zeta potential of a nanoparticle may be about −5 mV, about −10 MV, about −15 mV or about −20 mV. Further, in some embodiments, the zeta potential of a nanoparticle may be −5 mV, −10 mV, −15 mV or −20 mV.

The lipid nanoparticle may be any one described herein including those listed in the Examples, such as Example 1.

Cationic and/or Ionisable Lipid

Any of a variety of cationic lipids may be used in the lipid nanoparticles of the invention.

Cationic lipids which are useful in the present invention can be any of a number of lipid species which carry a net positive charge at physiological pH. Such lipids include, but are not limited to, N,N-dioleyl-N,N-dimethylammonium chloride (DODAC), 1,2-dioeoyloxy-3-(dimethylamino)propane (DODAP), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3.beta.-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 4-Hydroxybutyl)azanediyl)bis(hexane-6,1-diyl) bis(2-hexyldecanoate) (ALC-0315), 8-[(2-hydroxyethyl)[6-oxo-6-(undecyloxy)hexyl]amino]-octanoic acid,1-octylnonyl ester (SM-102) and mixtures thereof. A number of these lipids and related analogs have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are each herein incorporated by reference in their entirety for all purposes. Additionally, a number of commercial preparations of cationic lipids are available and can be used in the present invention. These include, e.g., LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic liposomes comprising DOGS from Promega Corp., Madison, Wis., USA).

Additionally, cationic lipids of Formula I having the following structures are useful in the present invention.

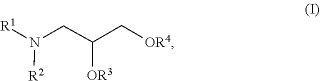

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In certain instances, R³ and R⁴ are both the same, i.e., R³ and R⁴ are both linoleyl (C₁₈), etc. In certain other instances, R³ and R⁴ are different, i.e., R³ is tetradectrienyl (C₁₄) and R⁴ is linoleyl (C₁₈). In a preferred embodiment, the cationic lipid of Formula I is symmetrical, i.e., R³ and R⁴ are both the same. In another preferred embodiment, both R³ and R⁴ comprise at least two sites of unsaturation. In some embodiments, R³ and R⁴ are independently selected from the group consisting of dodecadienyl, tetradecadienyl, hexadecadienyl, linoleyl, and icosadienyl. In a preferred embodiment, R³ and R⁴ are both linoleyl. In some embodiments, R³ and R⁴ comprise at least three sites of unsaturation and are independently selected from, e.g., dodecatrienyl, tetradectrienyl, hexadecatrienyl, linolenyl, and icosatrienyl. In particularly preferred embodiments, the cationic lipid of Formula I is 1,2-dilinoleyloxy-N,N-dimethylaminopropane (DLinDMA) or 1,2-dilinolenyloxy-N,N-dimethylaminopropane (DLenDMA).

Furthermore, cationic lipids of Formula II having the following structures are useful in the present invention.

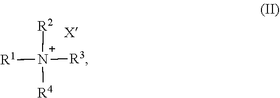

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In certain instances, R³ and R⁴ are both the same, i.e., R³ and R⁴ are both linoleyl (C₁₈), etc. In certain other instances, R³ and R⁴ are different, i.e., R³ is tetradectrienyl (C₁₄) and R⁴ is linoleyl (C₁₈). In a preferred embodiment, the cationic lipids of the present invention are symmetrical, i.e., R³ and R⁴ are both the same. In another preferred embodiment, both R³ and R⁴ comprise at least two sites of unsaturation. In some embodiments, R³ and R⁴ are independently selected from the group consisting of dodecadienyl, tetradecadienyl, hexadecadienyl, linoleyl, and icosadienyl. In a preferred embodiment, R³ and R⁴ are both linoleyl. In some embodiments, R³ and R⁴ comprise at least three sites of unsaturation and are independently selected from, e.g., dodecatrienyl, tetradectrienyl, hexadecatrienyl, linolenyl, and icosatrienyl.

Moreover, cationic lipids of Formula III having the following structures (or salts thereof) are useful in the present invention.

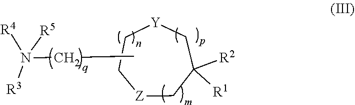

Wherein R¹ and R² are either the same or different and independently optionally substituted C₁₂-C₂₄ alkyl, optionally substituted C₁₂-C₂₄ alkenyl, optionally substituted C₁₂-C₂₄ alkynyl, or optionally substituted C₁₂-C₂₄ acyl; R³ and R⁴ are either the same or different and independently optionally substituted C₁-C₆ alkyl, optionally substituted C₁-C₆ alkenyl, or optionally substituted C₁-C₅ alkynyl or R³ and R⁴ may join to form an optionally substituted heterocyclic ring of 4 to 6 carbon atoms and 1 or 2 heteroatoms chosen from nitrogen and oxygen; R⁵ is either absent or hydrogen or C₁-C₆ alkyl to provide a quaternary amine; m, n, and p are either the same or different and independently either 0 or 1 with the proviso that m, n, and p are not simultaneously 0; q is 0, 1, 2, 3, or 4; and Y and Z are either the same or different and independently O, S, or NH.

In some embodiments, the cationic lipid of Formula III is 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), or mixtures thereof. In preferred embodiments, the cationic lipid of Formula III is DLin-K-C2-DMA (XTC2).

Preferably, the cationic lipid is DODAP, DLin-DMA, DLin-K-DMA, DLin-K2-DMA DLin-MC3-DMA.

The cationic lipid typically comprises from about 40 mol % to about 60 mol %, from about 40 mol % to about 55 mol %, from about 40 mol % to about 50 mol %, from about 40 mol % to about 45 mol %, from about 45 mol % to about 60 mol %, from about 50 mol % to about 60 mol %, or from about 55 mol % to about 60 mol % of the total lipid present in the particle.

The cationic lipid typically comprises about 40, 41, 42, 43, 44, 45, 46, 47, 48, 49, 50, 51, 52, 53, 54, 55, 56, 57, 58, 59 or 60 mol % of the total lipid present in the particle.

### Phospholipid

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

The lipid component of a lipid nanoparticle or composition may include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to Formula (IV):

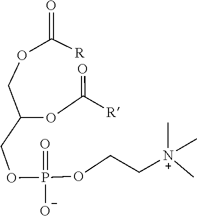

in which represents a phospholipid moiety and R and R′ represent fatty acid moieties with or without unsaturation that may be the same or different. A phospholipid moiety may be selected from the non-limiting group consisting of:
phosphatidyl choline,
phosphatidyl ethanolamine,
phosphatidyl glycerol,
phosphatidyl serine,
phosphatidic acid,
2-lysophosphatidyl choline, and
a sphingomyelin.

A fatty acid moiety may be selected from the non-limiting group consisting of:
lauric acid,
myristic acid,
myristoleic acid,
palmitic acid,
palmitoleic acid,
stearic acid,
oleic acid,
linoleic acid,
alpha-linolenic acid,
erucic acid,
phytanoic acid,
arachidic acid,
arachidonic acid,
eicosapentaenoic acid,
behenic acid,
docosapentaenoic acid, and
docosahexaenoic acid.

Non-natural species including natural species with modifications and substitutions including branch-ing, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Contemplated are phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

In some embodiments, a nanoparticle composition includes DSPC. In certain embodiments, a nanoparticle composition includes DOPE. In some embodiments, a nanoparticle composition includes both DSPC and DOPE.

The phospholipid typically comprises from about 5 mol % to about 20 mol %, from about 5 mol % to about 15 mol %, from about 5 mol % to about 10 mol %, from about 10 mol % to about 20 mol %, or from about 15 mol % to about 20 mol % of the total lipid present in the particle.

The phospholipid typically comprises from 5 mol % to 20 mol %, from 5 mol % to mol %, from 5 mol % to 10 mol %, from 10 mol % to 20 mol %, or from 15 mol % to mol % of the total lipid present in the particle.

### Structural Lipid

The lipid component of a nanoparticle composition may include one or more structural lipids. Structural lipids can be selected from the group consisting of, but are not limited to, cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof. Further, the structural lipid may be squalene, squalene or combination thereof.

The structural lipid may include lipids containing geranyl acetate, farnesyl acetate or geranyl-geranyl, or ether, ester, or other derivatives.

The structural lipid typically comprises from about 30 mol % to about 50 mol %, from about 30 mol % to about 45 mol %, from about 30 mol % to about 40 mol %, from about 30 mol % to about 35 mol %, from about 35 mol % to about 50 mol %, from about mol % to about 50 mol %, or from about 45 mol % to about 50 mol % of the total lipid present in the particle.

The structural lipid typically comprises from 30 mol % to 50 mol %, from 30 mol % to 45 mol %, from 30 mol % to 40 mol %, from 30 mol % to 35 mol %, from 35 mol % to 50 mol %, from 40 mol % to 50 mol %, or from 45 mol % to 50 mol % of the total lipid present in the particle.

### PEGylated Lipid

The lipid component of a lipid nanoparticle or composition may include one or more PEG or PEG-modified lipids. Such species may be alternately referred to as PEGylated lipids. As used herein, a “PEG lipid” or “PEGylated lipid” refers to a lipid comprising a polyethylene glycol component. A PEG lipid may be selected from the non-limiting group consisting of:
PEG-modified phosphatidylethanolamines,
PEG-modified phosphatidic acids,
PEG-modified ceramides,
PEG-modified dialkylamines,
PEG-modified diacylglycerols,
PEG-modified dialkylglycerols, and
mixtures thereof.

For example, a PEG lipid may be PEG-c-DOMG, PEG-DMG, PEG-DLPE, PEG-DMPE, PEG-DPPC, or a PEG-DSPE lipid.

In another embodiment, the PEGylated lipid may be 1,2-dimyristoyl-rac-glycero-3-methoxypolyethylene glycol-2000, also known as DMG-PEG.

In another embodiment, the PEGylated lipid may be 2-[(polyethylene glycol)-2000]-N,N-ditetradecylacetamide (ALC-0159).

The PEGylated lipid may have a PEG component that has a molecular weight of any molecular mass as practically desired, including but not limited to, from about 100 Daltons (Da) to 10,000 Da or more as desired (including but not limited to, sometimes 0.1-10 kDa). The molecular weight of PEG may be of a wide range, including but not limited to, between about 100 Da and about 10,000 Da or more. PEG may be between about 100 Da and about 100,000 Da, including but not limited to 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, PEG is between about 100 Da and 10,000 Da, between about 1000 Da and 9,000 Da, between about 1000 Da and 8,000 Da, between about 1000 Da and 7,000 Da, between about 1000 Da and 6,000 Da, between about 1000 Da and 5,000 Da, between about 1000 Da and 4,000 Da, between about 1000 Da and 3,000 Da, or between about 1000 Da and 2,000 Da. In some embodiments, PEG is between about 1,000 Da and 5,000 Da. In some embodiments, PEG is between about 2,000 Da and 5,000 Da.

The PEGylated lipid typically comprises from about 0.05 mol % to about 0.5 mol %, from about 0.06 mol % to about 0.5 mol %, from about 0.07 mol % to about 0.5 mol %, from about 0.08 mol % to about 0.5 mol %, from about 0.09 mol % to about 0.5 mol %, from about 0.1 mol % to about 0.5 mol %, from about 0.15 mol % to about 0.5 mol %, from about 0.2 mol % to about 0.5 mol %, from about 0.25 mol % to about 0.5 mol %, from about 0.3 mol % to about 0.5 mol %, from about 0.3 mol % to about 0.5 mol %, from about 0.35 mol % to about 0.5 mol %, from about 0.4 mol % to about 0.5 mol %, from about 0.45 mol % to about 0.5 mol %, from about 0.05 mol % to about 0.45 mol %, from about 0.05 mol % to about 0.4 mol %, from about 0.05 mol % to about 0.35 mol %, from about 0.05 mol % to about 0.3 mol %, from about 0.05 mol % to about 0.25 mol %, from about 0.05 mol % to about 0.2 mol %, from about 0.05 mol % to about 0.15 mol %, from about 0.05 mol % to about 0.1 mol %, from about 0.05 mol % to about 0.09 mol %, from about 0.05 mol % to about 0.08 mol %, from about 0.05 mol % to about 0.07 mol %, or from about 0.05 mol % to about 0.06 mol % of the total lipid present in the particle.

The PEGylated lipid typically comprises from 0.05 mol % to 0.5 mol %, from 0.06 mol % to 0.5 mol %, from 0.07 mol % to 0.5 mol %, from 0.08 mol % to 0.5 mol %, from 0.09 mol % to 0.5 mol %, from 0.1 mol % to 0.5 mol %, from 0.15 mol % to 0.5 mol %, from 0.2 mol % to 0.5 mol %, from 0.25 mol % to 0.5 mol %, from 0.3 mol % to 0.5 mol %, from 0.3 mol % to 0.5 mol %, from 0.35 mol % to 0.5 mol %, from 0.4 mol % to 0.5 mol %, from 0.45 mol % to 0.5 mol %, from 0.05 mol % to 0.45 mol %, from 0.05 mol % to 0.4 mol %, from 0.05 mol % to 0.35 mol %, from 0.05 mol % to 0.3 mol %, from 0.05 mol % to 0.25 mol %, from 0.05 mol % to 0.2 mol %, from 0.05 mol % to 0.15 mol %, from 0.05 mol % to 0.1 mol %, from 0.05 mol % to 0.09 mol %, from 0.05 mol % to 0.08 mol %, from 0.05 mol % to 0.07 mol %, or from 0.05 mol % to 0.06 mol % of the total lipid present in the particle.

The PEGylated lipid typically comprises 0.05 mol %, 0.06 mol %, 0.07 mol %, 0.08 mol %, 0.09 mol %, 0.1 mol %, 0.15 mol %, 0.2 mol %, 0.25 mol %, 0.3 mol %, 0.35 mol %, 0.4 mol %, or 0.45 mol % of the total lipid present in the particle.

As used herein, mole % and mol % are used interchangeably.

### Active Agents

Lipid nanoparticles may include one or more therapeutic and/or prophylactic agents. The disclosure features methods of delivering a therapeutic and/or prophylactic agents to a mammalian cell or organ, optionally producing a polypeptide of interest in a mammalian cell, and treating a disease or disorder in a mammal in need thereof comprising administering to a mammal and/or contacting a mammalian cell with a lipid nanoparticle including a therapeutic and/or prophylactic agent.

Therapeutic and/or prophylactic agents include biologically active substances and are alternately referred to as “active agents.” A therapeutic and/or prophylactic agent may be a substance that, once delivered to a cell or organ, brings about a desirable change in the cell, organ, or other bodily tissue or system. Such agents may be useful in the treatment of one or more diseases, disorders, or conditions. In some embodiments, a therapeutic and/or prophylactic agent is a small molecule drug useful in the treatment of a particular disease, disorder, or condition. Examples of drugs useful in the nanoparticle compositions include, but are not limited to, antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cystine arabinoside, anthracyclines, alkylative agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetolol), antihypertensive agents (e.g., clonidine and hydralazine), anti-depressants (e.g., imipramine, amitriptyline, and doxepim), anti-conversants (e.g., phenytoin), antihistamines (e.g., diphenhydramine, chlorphenirimine, and promethazine), antibiotic/antibacterial agents (e.g., gen-tamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

The present invention provides novel lipid nanoparticles comprising one or more active agents, methods of making the lipid particles, and methods of delivering and/or administering the lipid nanoparticles (e.g., for the treatment of a disease or disorder).

In one aspect, the present invention provides a lipid nanoparticle comprising:
(a) an active agent;
(b) a cationic and/or ionisable lipid comprising from about 40 mol % to about 60 mol % of the total lipid present in the nanoparticle;
(c) a phospholipid comprising from about 5 mol % to about 20 mol % of the total lipid present in the nanoparticle;
(d) a structural lipid comprising from about 30 mol % to about 50 mol % of the total lipid present in the nanoparticle;
(e) a PEGylated lipid comprising from about 0.05 mol % to less than 0.5 mol % of the total lipid present in the nanoparticle.

As used herein, (b), (c), (d) and (e) may be referred to as the “lipid component”.

In certain embodiments, the active agent or therapeutic agent is fully encapsulated within the lipid portion of the lipid particle such that the active agent in the lipid nanoparticle is resistant in aqueous solution to enzymatic degradation, e.g., by a nuclease or protease. In certain other embodiments, the lipid nanoparticles are substantially non-toxic to mammals such as humans.

In some embodiments, the active agent or therapeutic agent comprises a nucleic acid. In certain instances, the nucleic acid comprises an interfering RNA molecule such as, e.g., an siRNA, aiRNA, miRNA, or mixtures thereof. In certain other instances, the nucleic acid comprises single-stranded or double-stranded DNA, RNA, or a DNA/RNA hybrid such as, e.g., an antisense oligonucleotide, a ribozyme, a plasmid, an immunostimulatory oligonucleotide, or mixtures thereof.

The amount of mRNA in a lipid nanoparticle may depend on the size, sequence, and other characteristics of the mRNA. The amount of mRNA in a lipid nanoparticle may also depend on the size, composition, desired target, and other characteristics of the lipid nanoparticle. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy).

In some embodiments, the wt/wt ratio of the lipid component to the mRNA in the nanoparticle composition is from about 5:1 to about 50:1. In certain embodiments, the wt/wt ratio is from about 10:1 to about 40:1.

In some embodiments, the one or more mRNAs, lipids, and amounts thereof may be selected to provide a specific N:P ratio. The N:P ratio of the composition refers to the molar ratio of nitrogen atoms in one or more lipids to the number of phosphate groups in an mRNA. In general, a lower N:P ratio is preferred. The one or more mRNA, lipids, and amounts thereof may be selected to provide an N:P ratio from about 2:1 to about 8:1, such as 2:1, 3:1, 4:1, 5:1, 6:1, 7:1, and 8:1. In certain embodiments, the N:P ratio may be from about 2:1 to about 5:1. In preferred embodiments, the N:P ratio may be about 4:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1.

In some embodiments, the N:P ratio of the nanoparticle composition is from about 2:1 to about 8:1. In particular embodiments, the N:P ratio is from about 2:1 to about 5:1. In preferred embodiments, the N:P ratio is about 4:1. In certain embodiments, the N:P ratio is from about 5:1 to about 8:1. For example, the N:P ratio may be about 5.0:1, about 5.5:1, about 5.67:1, about 6.0:1, about 6.5:1, or about 7.0:1.

In other embodiments, the active agent or therapeutic agent comprises a peptide or polypeptide. In certain instances, the peptide or polypeptide comprises an antibody such as, e.g., a polyclonal antibody, a monoclonal antibody, an antibody fragment; a humanized antibody, a recombinant antibody, a recombinant human antibody, a Primatized™ antibody, or mixtures thereof. In certain other instances, the peptide or polypeptide comprises a cytokine, a growth factor, an apoptotic factor, a differentiation-inducing factor, a cell-surface receptor, a ligand, a hormone, a small molecule (e.g., small organic molecule or compound), or mixtures thereof.

In some embodiments, the active agent is a therapeutic agent, or a salt or derivative thereof. Therapeutic agent derivatives may be therapeutically active themselves or they may be prodrugs, which become active upon further modification. Thus, in one embodiment, a therapeutic agent derivative retains some or all of the therapeutic activity as compared to the unmodified agent, while in another embodiment, a therapeutic agent derivative is a prodrug that lacks therapeutic activity, but becomes active upon further modification.

### A. Nucleic Acids

In certain embodiments, lipid nanoparticles of the present invention are associated with a nucleic acid, resulting in a nucleic acid-lipid nanoparticle (e.g., NALP). In some embodiments, the nucleic acid is fully encapsulated in the lipid particle. As used herein, the term “nucleic acid” includes any oligonucleotide or polynucleotide, with fragments containing up to 60 nucleotides generally termed oligonucleotides, and longer fragments termed polynucleotides.

In particular embodiments, oligonucleotides of the invention are from about 15 to about 60 nucleotides in length. Nucleic acid may be administered alone in the lipid particles of the invention, or in combination (e.g., co-administered) with lipid nanoparticles of the invention comprising peptides, polypeptides, or small molecules such as conventional drugs.

In the context of this invention, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Oligonucleotides are generally classified as deoxyribooligonucleotides or ribooligonucleotides. A deoxyribooligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose.

The nucleic acid that is present in a nucleic acid-lipid nanoparticle according to this invention includes any form of nucleic acid that is known. The nucleic acids used herein can be single-stranded DNA or RNA (e.g. pre-mRNA, mature mRNA, mRNA), or double-stranded DNA or RNA, or DNA-RNA hybrids. Examples of double-stranded DNA are described herein and include, e.g., structural genes, genes including control and termination regions, and self-replicating systems such as viral or plasmid DNA. Examples of double-stranded RNA are described herein and include, e.g., siRNA and other RNAi agents such as aiRNA and pre-miRNA. Single-stranded nucleic acids include, e.g., antisense oligonucleotides, ribozymes, mature miRNA, and triplex-forming oligonucleotides.

The mRNA contained in or encapsulated by the lipid nanoparticles of the present invention may encoding a polypeptide of interest. Preferably, the mRNA is capable of being translated in the cell to produce the polypeptide of interest. The polypeptide of interest may be any antigenic or immunogenic polypeptide, such as that that is used to stimulate the humoral (eg B cell or T cell) immune system. The polypeptide of interest may be useful for the therapeutic or prophylactic immunisation of a mammal, preferably human. The polypeptide of interest may useful for providing a therapeutic or prophylactic effect against a disease or condition, preferably wherein the condition is an infection. The infection may be by any microorganism, such as a bacteria, virus, fungi or protozoa. The virus may be any virus including but not limited to coronavirus, preferably SARS-CoV or SARS-CoV-2.

Nucleic acids of the invention may be of various lengths, generally dependent upon the particular form of nucleic acid. For example, in particular embodiments, plasmids or genes may be from about 100 to about 100,000 nucleotide residues in length. In particular embodiments, oligonucleotides may range from about 10 to about 100 nucleotides in length. In various related embodiments, oligonucleotides, both single-stranded, double-stranded, and triple-stranded, may range in length from about to about 60 nucleotides, from about 15 to about 60 nucleotides, from about 20 to about 50 nucleotides, from about 15 to about 30 nucleotides, or from about 20 to about nucleotides in length.

In particular embodiments, an oligonucleotide (or a strand thereof) of the invention specifically hybridizes to or is complementary to a target polynucleotide sequence. The terms “specifically hybridizable” and “complementary” as used herein indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. In preferred embodiments, an oligonucleotide is specifically hybridizable when binding of the oligonucleotide to the target sequence interferes with the normal function of the target sequence to cause a loss of utility or expression therefrom, and there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under conditions in which the assays are conducted. Thus, the oligonucleotide may include 1, 2, 3, or more base substitutions as compared to the region of a gene or mRNA sequence that it is targeting or to which it specifically hybridizes.

1. siRNA

The siRNA component of the nucleic acid-lipid particles of the present invention is capable of silencing the expression of a target gene of interest. Each strand of the siRNA duplex is typically about 15 to about 60 nucleotides in length, preferably about 15 to about 30 nucleotides in length. In certain embodiments, the siRNA comprises at least one modified nucleotide. The modified siRNA is generally less immunostimulatory than a corresponding unmodified siRNA sequence and retains RNAi activity against the target gene of interest. In some embodiments, the modified siRNA contains at least one 2′OMe purine or pyrimidine nucleotide such as a 2′OMe-guanosine, 2′OMe-uridine, 2′OMe-adenosine, and/or 2′OMe-cytosine nucleotide. In preferred embodiments, one or more of the uridine and/or guanosine nucleotides are modified. The modified nucleotides can be present in one strand (i.e., sense or antisense) or both strands of the siRNA. The siRNA sequences may have overhangs (e.g., 3′ or 5′ overhangs as described in Elbashir et al., Genes Dev., 15:188 (2001) or Nyilnen et al., Cell, 107:309 (2001)), or may lack overhangs (i.e., have blunt ends).

The modified siRNA generally comprises from about 1% to about 100% (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) modified nucleotides in the double-stranded region of the siRNA duplex. In certain embodiments, one, two, three, four, five, six, seven, eight, nine, ten, or more of the nucleotides in the double-stranded region of the siRNA comprise modified nucleotides.

In some embodiments, less than about 25% (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) of the nucleotides in the double-stranded region of the siRNA comprise modified nucleotides.

In other embodiments, from about 1% to about 25% (e.g., from about 1%-25%, 2%-25%, 3%-25%, 4%-25%, 5%-25%, 6%-25%, 7%-25%, 8%-25%, 9%-25%, 10%-25%, 11%-25%, 12%-25%, 13%-25%, 14%-25%, 15%-25%, 16%-25%, 17%-25%, 18%-25%, 19%-25%, 20%-25%, 21%-25%, 22%-25%, 23%-25%, 24%-25%, etc.) or from about 1% to about 20% (e.g., from about 1%-20%, 2%-20%, 3%-20%, 4%-20%, 5%-20%, 6%-20%, 7%-20%, 8%-20%, 9%-20%, 10%-20%, 11%-20%, 12%-20%, 13%-20%, 14%-20%, 15%-20%, 16%-20%, 17%-20%, 18%-20%, 19%-20%, 1%-19%, 2%-19%, 3%-19%, 4%-19%, 5%-19%, 6%-19%, 7%-19%, 8%-19%, 9%-19%, 10%-19%, 11%-19%, 12%-19%, 13%-19%, 14%-19%, 15%-19%, 16%-19%, 17%-19%, 18%-19%, 1%-18%, 2%-18%, 3%-18%, 4%-18%, 5%-18%, 6%-18%, 7%-18%, 8%-18%, 9%-18%, 10%-18%, 11%-18%, 12%-18%, 13%-18%, 14%-18%, 15%-18%, 16%-18%, 17%-18%, 1%-17%, 2%-17%, 3%-17%, 4%-17%, 5%-17%, 6%-17%, 7%-17%, 8%-17%, 9%-17%, 10%-17%, 11%-17%, 12%-17%, 13%-17%, 14%-17%, 15%-17%, 16%-17%, 1%-16%, 2%-16%, 3%-16%, 4%-16%, 5%-16%, 6%-16%, 7%-16%, 8%-16%, 9%-16%, 10%-16%, 11%-16%, 12%-16%, 13%-16%, 14%-16%, 15%-16%, 1%-15%, 2%-15%, 3%-15%, 4%-15%, 5%-15%, 6%-15%, 7%-15%, 8%-15%, 9%-15%, 10%-15%, 11%-15%, 12%-15%, 13%-15%, 14%-15%, etc.) of the nucleotides in the double-stranded region of the siRNA comprise modified nucleotides.

In further embodiments, e.g., when one or both strands of the siRNA are selectively modified at uridine and/or guanosine nucleotides, the resulting modified siRNA can comprise less than about 30% modified nucleotides (e.g., less than about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% modified nucleotides) or from about 1% to about 30% modified nucleotides (e.g., from about 1%-30%, 2%-30%, 3%-30%, 4%-30%, 5%-30%, 6%-30%, 7%-30%, 8%-30%, 9%-30%, 10%-30%, 11%-30%, 12%-30%, 13%-30%, 14%-30%, 15%-30%, 16%-30%, 17%-30%, 18%-30%, 19%-30%, 20%-30%, 21%-30%, 22%-30%, 23%-30%, 24%-30%, 25%-30%, 26%-30%, 27%-30%, 28%-30%, or 29%-30% modified nucleotides).

a. Selection of siRNA Sequences

Suitable siRNA sequences can be identified using any means known in the art. Typically, the methods described in Elbashir et al., Nature, 411:494-498 (2001) and Elbashir et al., EMBO J., 20:6877-6888 (2001) are combined with rational design rules set forth in Reynolds et al., Nature Biotech., 22(3):326-330 (2004).

Generally, the nucleotide sequence 3′ of the AUG start codon of a transcript from the target gene of interest is scanned for dinucleotide sequences (e.g., AA, NA, CC, GG, or UU, wherein N=C, G, or U) (see, e.g., Elbashir et al., EMBO J., 20:6877-6888 (2001)). The nucleotides immediately 3′ to the dinucleotide sequences are identified as potential siRNA sequences (i.e., a target sequence or a sense strand sequence). Typically, the 19, 21, 23, 25, 27, 29, 31, 33, 35, or more nucleotides immediately 3′ to the dinucleotide sequences are identified as potential siRNA sequences. In some embodiments, the dinucleotide sequence is an AA or NA sequence and the 19 nucleotides immediately 3′ to the AA or NA dinucleotide are identified as potential siRNA sequences. siRNA sequences are usually spaced at different positions along the length of the target gene. To further enhance silencing efficiency of the siRNA sequences, potential siRNA sequences may be analyzed to identify sites that do not contain regions of homology to other coding sequences, e.g., in the target cell or organism. For example, a suitable siRNA sequence of about 21 base pairs typically will not have more than 16-17 contiguous base pairs of homology to coding sequences in the target cell or organism. If the siRNA sequences are to be expressed from an RNA Pol III promoter, siRNA sequences lacking more than 4 contiguous A's or T's are selected.

Once a potential siRNA sequence has been identified, a complementary sequence (i.e., an antisense strand sequence) can be designed. A potential siRNA sequence can also be analyzed using a variety of criteria known in the art. For example, to enhance their silencing efficiency, the siRNA sequences may be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. siRNA design tools that incorporate algorithms that assign suitable values of each of these features and are useful for selection of siRNA can be found at, e.g., http://boz094.ust.hk/RNAi/siRNA. One of skill in the art will appreciate that sequences with one or more of the foregoing characteristics may be selected for further analysis and testing as potential siRNA sequences.

Additionally, potential siRNA sequences with one or more of the following criteria can often be eliminated as siRNA: (1) sequences comprising a stretch of 4 or more of the same base in a row; (2) sequences comprising homopolymers of Gs (i.e., to reduce possible non-specific effects due to structural characteristics of these polymers; (3) sequences comprising triple base motifs (e.g., GGG, CCC, AAA, or ITT); (4) sequences comprising stretches of 7 or more G/Cs in a row; and (5) sequences comprising direct repeats of 4 or more bases within the candidates resulting in internal fold-back structures. However, one of skill in the art will appreciate that sequences with one or more of the foregoing characteristics may still be selected for further analysis and testing as potential siRNA sequences.

In some embodiments, potential siRNA sequences may be further analyzed based on siRNA duplex asymmetry as described in, e.g., Khvorova et al., Cell, 115:209-216 (2003); and Schwarz at al., Cell, 115:199-208 (2003). In other embodiments, potential siRNA sequences may be further analyzed based on secondary structure at the target site as described in, e.g., Luo et al., Biophys. Res. Commun., 318:303-310 (2004). For example, secondary structure at the target site can be modeled using the Mfold algorithm (available at http://www.bioinfo.rpi.edu/applications/mfold/rna/forml.cgi) to select siRNA sequences which favor accessibility at the target site where less secondary structure in the form of base-pairing and stem-loops is present.

Once a potential siRNA sequence has been identified, the sequence can be analyzed for the presence of any immunostimulatory properties, e.g., using an in vitro cytokine assay or an in vivo animal model. Motifs in the sense and/or antisense strand of the siRNA sequence such as GU-rich motifs (e.g., 5′-GU-3′,5′-UGU-3′,5′-GUGU-3′,5′-UGUGU-3′, etc.) can also provide an indication of whether the sequence may be immunostimulatory. Once an siRNA molecule is found to be immunostimulatory, it can then be modified to decrease its immunostimulatory properties as described herein. As a non-limiting example, an siRNA sequence can be contacted with a mammalian responder cell under conditions such that the cell produces a detectable immune response to determine whether the siRNA is an immunostimulatory or a non-immunostimulatory siRNA. The mammalian responder cell may be from a naïve mammal (i.e., a mammal that has not previously been in contact with the gene product of the siRNA sequence). The mammalian responder cell may be, e.g., a peripheral blood mononuclear cell (PBMC), a macrophage, and the like. The detectable immune response may comprise production of a cytokine or growth factor such as, e.g., TNF-α, IFN-α, IFN-β, IFN-γ, IL-6, IL-12, or a combination thereof. An siRNA molecule identified as being immunostimulatory can then be modified to decrease its immunostimulatory properties by replacing at least one of the nucleotides on the sense and/or antisense strand with modified nucleotides. For example, less than about 30% (e.g., less than about 30%, 25%, 20%, 15%, 10%, or 5%) of the nucleotides in the double-stranded region of the siRNA duplex can be replaced with modified nucleotides such as 2′OMe nucleotides. The modified siRNA can then be contacted with a mammalian responder cell as described above to confirm that its immunostimulatory properties have been reduced or abrogated.

Suitable in vitro assays for detecting an immune response include, but are not limited to, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “Western blot” method of Gordon et al. (U.S. Pat. No. 4,452,901); immunoprecipitation of labeled ligand (Brown et al., J. Biol. Chem., 255:4980-4983 (1980)); enzyme-linked immunosorbent assays (ELISA) as described, for example, by Raines et al., J. Biol. Chem., 257:5154-5160 (1982); immunocytochemical techniques, including the use of fluorochromes (Brooks et al., Clin. Exp. Immunol., 39:477 (1980)); and neutralization of activity (Bowen-Pope et al., Proc. Natl. Acad. Sci. USA, 81:2396-2400 (1984)). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876. The disclosures of these references are herein incorporated by reference in their entirety for all purposes.

A non-limiting example of an in vivo model for detecting an immune response includes an in vivo mouse cytokine induction assay as described in, e.g., Judge et al., Mol. Ther., 13:494-505 (2006). In certain embodiments, the assay that can be performed as follows: (1) siRNA can be administered by standard intravenous injection in the lateral tail vein; (2) blood can be collected by cardiac puncture about 6 hours after administration and processed as plasma for cytokine analysis; and (3) cytokines can be quantified using sandwich ELISA kits according to the manufacturer's instructions (e.g., mouse and human IFN-α (PBL Biomedical; Piscataway, N.J.); human IL-6 and TNF-α (eBioscience; San Diego, Calif.); and mouse IL-6, TNF-α, and IFN-γ (BD Biosciences; San Diego, Calif.)).

Monoclonal antibodies that specifically bind cytokines and growth factors are commercially available from multiple sources and can be generated using methods known in the art (see, e.g., Kohler et al., Nature, 256: 495-497 (1975) and Harlow and Lane, ANTIBODIES, A LABORATORY MANUAL, Cold Spring Harbor Publication, New York (1999)). Generation of monoclonal antibodies has been previously described and can be accomplished by any means known in the art (Buhring et al., in Hybridoma, Vol. 10, No. 1, pp. 77-78 (1991)). In some methods, the monoclonal antibody is labeled (e.g., with any composition detectable by spectroscopic, photochemical, biochemical, electrical, optical, or chemical means) to facilitate detection.

b. Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. The siRNA sequences may have overhangs (e.g., 3′ or 5′ overhangs as described in Elbashir et al., Genes Dev., 15:188 (2001) or Nyksnen et al., Cell, 107:309 (2001), or may lack overhangs (i.e., to have blunt ends).

An RNA population can be used to provide long precursor RNAs, or long precursor RNAs that have substantial or complete identity to a selected target sequence can be used to make the siRNA. The RNAs can be isolated from cells or tissue, synthesized, and/or cloned according to methods well known to those of skill in the art. The RNA can be a mixed population (obtained from cells or tissue, transcribed from cDNA, subtracted, selected, etc.), or can represent a single target sequence. RNA can be naturally occurring (e.g., isolated from tissue or cell samples), synthesized in vitro (e.g., using T7 or SP6 polymerase and PCR products or a cloned cDNA), or chemically synthesized.

To form a long dsRNA, for synthetic RNAs, the complement is also transcribed in vitro and hybridized to form a dsRNA. If a naturally occurring RNA population is used, the RNA complements are also provided (e.g., to form dsRNA for digestion by E. coli RNAse Ill or Dicer), e.g., by transcribing cDNAs corresponding to the RNA population, or by using RNA polymerases. The precursor RNAs are then hybridized to form double stranded RNAs for digestion. The dsRNAs can be directly administered to a subject or can be digested in vitro prior to administration.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Preferably, siRNA are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope of this invention. Suitable reagents for oligonucleotide synthesis, methods for RNA deprotection, and methods for RNA purification are known to those of skill in the art.

siRNA molecules can also be synthesized via a tandem synthesis technique, wherein both strands are synthesized as a single continuous oligonucleotide fragment or strand separated by a cleavable linker that is subsequently cleaved to provide separate fragments or strands that hybridize to form the siRNA duplex. The linker can be a polynucleotide linker or a non-nucleotide linker. The tandem synthesis of siRNA can be readily adapted to both multiwell/multiplate synthesis platforms as well as large scale synthesis platforms employing batch reactors, synthesis columns, and the like. Alternatively, siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection. In certain other instances, siRNA molecules can be synthesized as a single continuous oligonucleotide fragment, where the self-complementary sense and antisense regions hybridize to form an siRNA duplex having hairpin secondary structure.

c. Modifying siRNA Sequences

In certain aspects, siRNA molecules comprise a duplex having two strands and at least one modified nucleotide in the double-stranded region, wherein each strand is about 15 to about 60 nucleotides in length. Advantageously, the modified siRNA is less immunostimulatory than a corresponding unmodified siRNA sequence, but retains the capability of silencing the expression of a target sequence. In preferred embodiments, the degree of chemical modifications introduced into the siRNA molecule strikes a balance between reduction or abrogation of the immunostimulatory properties of the siRNA and retention of RNAi activity. As a non-limiting example, an siRNA molecule that targets a gene of interest can be minimally modified (e.g., less than about 30%, 25%, 20%, 15%, 10%, or 5% modified) at selective uridine and/or guanosine nucleotides within the siRNA duplex to eliminate the immune response generated by the siRNA while retaining its capability to silence target gene expression.

Examples of modified nucleotides suitable for use in the invention include, but are not limited to, ribonucleotides having a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl group. Modified nucleotides having a Northern conformation such as those described in, e.g., Saenger, Principles of Nucleic Acid Structure, Springer-Verlag Ed. (1984), are also suitable for use in siRNA molecules. Such modified nucleotides include, without limitation, locked nucleic acid (LNA) nucleotides (e.g., 2′-O, 4′-C-methylene-(D-ribofuranosyl) nucleotides), 2′-O-(2-methoxyethyl) (MOE) nucleotides, 2′-methyl-thio-ethyl nucleotides, 2′-deoxy-2′-fluoro (2′F) nucleotides, 2′-deoxy-2′-chloro (2′Cl) nucleotides, and 2′-azido nucleotides. In certain instances, the siRNA molecules described herein include one or more G-clamp nucleotides. A G-clamp nucleotide refers to a modified cytosine analog wherein the modifications confer the ability to hydrogen bond both Watson-Crick and Hoogsteen faces of a complementary guanine nucleotide within a duplex (see, e.g., Lin et al., J Am. Chem. Soc., 120:8531-8532 (1998)). In addition, nucleotides having a nucleotide base analog such as, for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (see, e.g., Loakes, Nucl. Acids Res., 29:2437-2447 (2001)) can be incorporated into siRNA molecules.

In certain embodiments, siRNA molecules may further comprise one or more chemical modifications such as terminal cap moieties, phosphate backbone modifications, and the like. Examples of terminal cap moieties include, without limitation, inverted deoxy abasic residues, glyceryl modifications, 4′,5′-methylene nucleotides, 1-(3-D-erythrofuranosyl) nucleotides, 4′-thio nucleotides, carbocyclic nucleotides, 1,5-anhydrohexitol nucleotides, L-nucleotides, α-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al., Tetrahedron 49:1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al., Nucleic Acid Analogues: Synthesis and Properties, in Modern Synthetic Methods, VCH, 331-417 (1995); Mesmaeker et al., Novel Backbone Replacements for Oligonucleotides, in Carbohydrate Modifications in Antisense Research, ACS, 24-39 (1994)). Such chemical modifications can occur at the 5′-end and/or 3′-end of the sense strand, antisense strand, or both strands of the siRNA. The disclosures of these references are herein incorporated by reference in their entirety for all purposes.

In some embodiments, the sense and/or antisense strand of the siRNA molecule can further comprise a 3′-terminal overhang having about 1 to about 4 (e.g., 1, 2, 3, or 4) 2′-deoxy ribonucleotides and/or any combination of modified and unmodified nucleotides. Additional examples of modified nucleotides and types of chemical modifications that can be introduced into siRNA molecules are described, e.g., in UK Patent No. GB 2,397,818 B and U.S. Patent Publication Nos. 20040192626, 20050282188, and 20070135372, the disclosures of which are herein incorporated by reference in their entirety for all purposes.

The siRNA molecules described herein can optionally comprise one or more non-nucleotides in one or both strands of the siRNA. As used herein, the term “non-nucleotide” refers to any group or compound that can be incorporated into a nucleic acid chain in the place of one or more nucleotide units, including sugar and/or phosphate substitutions, and allows the remaining bases to exhibit their activity. The group or compound is abasic in that it does not contain a commonly recognized nucleotide base such as adenosine, guanine, cytosine, uracil, or thymine and therefore lacks a base at the 1′-position.

In other embodiments, chemical modification of the siRNA comprises attaching a conjugate to the siRNA molecule. The conjugate can be attached at the 5′ and/or 3′-end of the sense and/or antisense strand of the siRNA via a covalent attachment such as, e.g., a biodegradable linker. The conjugate can also be attached to the siRNA, e.g., through a carbamate group or other linking group (see, e.g., U.S. Patent Publication Nos. 20050074771, 20050043219, and 20050158727). In certain instances, the conjugate is a molecule that facilitates the delivery of the siRNA into a cell. Examples of conjugate molecules suitable for attachment to siRNA include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Yet other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples include the hydrophobic group, membrane active compound, cell penetrating compound, cell targeting signal, interaction modifier, and steric stabilizer conjugate molecules described in U.S. Patent Publication No. 20040167090. Further examples include the conjugate molecules described in U.S. Patent Publication No. 20050239739. The type of conjugate used and the extent of conjugation to the siRNA molecule can be evaluated for improved pharmacokinetic profiles, bioavailability, and/or stability of the siRNA while retaining RNAi activity. As such, one skilled in the art can screen siRNA molecules having various conjugates attached thereto to identify ones having improved properties and full RNAi activity using any of a variety of well-known in vitro cell culture or in vivo animal models. The disclosures of the above-described patent documents are herein incorporated by reference in their entirety for all purposes.

d. Target Genes

The siRNA component of the nucleic acid-lipid particles described herein can be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation (e.g., cancer), angiogenic genes, immunomodulator genes such as those associated with inflammatory and autoimmune responses.

Genes associated with metabolic diseases and disorders (e.g., disorders in which the liver is the target and liver diseases and disorders) include, for example, genes expressed in dyslipidemia (e.g., liver X receptors such as LXRα and LXRβ (Genback Accession No. NM_007121), farnesoid X receptors (FXR) (Genbank Accession No. NM_005123), sterol-regulatory element binding protein (SREBP), site-1 protease (SIP), 3-hydroxy-3-methylglutaryl coenzyme-A reductase (HMG coenzyme-A reductase), apolipoprotein B (ApoB) (Genbank Accession No. NM_000384), apolipoprotein CIII (ApoC3) (Genbank Accession Nos. NM_000040 and NG_008949 REGION: 5001.8164), and apolipoprotein E (ApoE) (Genbank Accession Nos. NM_000041 and NG_007084 REGION: 5001.8612)); and diabetes (e.g., glucose 6-phosphatase) (see, e.g., Forman et al., Cell, 81:687 (1995); Seol et al., Mol. Endocrinol., 9:72 (1995), Zavacki et al., Proc. Natl. Acad. Sci. USA, 94:7909 (1997); Sakai et al., Cell, 85:1037-1046 (1996); Duncan et al., J. Biol. Chem., 272:12778-12785 (1997); Willy et al., Genes Dev., 9:1033-1045 (1995); Lehmann et al., J. Biol. Chem., 272:3137-3140 (1997); Janowski et al., Nature, 383:728-731 (1996); and Peet et al., Cell, 93:693-704 (1998)). One of skill in the art will appreciate that genes associated with metabolic diseases and disorders (e.g., diseases and disorders in which the liver is a target and liver diseases and disorders) include genes that are expressed in the liver itself as well as and genes expressed in other organs and tissues. Silencing of sequences that encode genes associated with metabolic diseases and disorders can conveniently be used in combination with the administration of conventional agents used to treat the disease or disorder. Non-limiting examples of siRNA molecules targeting the ApoB gene include those described in U.S. Patent Publication No. 20060134189, the disclosure of which is herein incorporated by reference in its entirety for all purposes. Non-limiting examples of siRNA molecules targeting the ApoC3 gene include those described in U.S. Provisional Application No. 61/147,235, filed Jan. 26, 2009, the disclosure of which is herein incorporated by reference in its entirety for all purposes.

Examples of gene sequences associated with tumorigenesis and cell transformation (e.g., cancer or other neoplasia) include mitotic kinesins such as Eg5 (KSP, KIF11; Genbank Accession No. NM_004523); serine/threonine kinases such as polo-like kinase 1 (PLK-1) (Genbank Accession No. NM_005030; Barr et al., Nat. Rev. Mol. Cell Biol., 5:429-440 (2004)); tyrosine kinases such as WEE1 (Genbank Accession Nos. NM_003390 and NM_001143976); inhibitors of apoptosis such as XIAP (Genbank Accession No. NM_001167); COP9 signalosome subunits such as CSN1, CSN2, CSN3, CSN4, CSN5 (JAB1; Genbank Accession No. NM_006837); CSN6, CSN7A, CSN7B, and CSN8; ubiquitin ligases such as COP1 (RFWD2; Genbank Accession Nos. NM_022457 and NM_001001740); and histone deacetylases such as HDAC1, HDAC2 (Genbank Accession No. NM 001527), HDAC3, HDAC4, HDAC5, HDAC6, HDAC7, FIDAC8, HDAC9, etc. Non-limiting examples of siRNA molecules targeting the Eg5 and XIAP genes include those described in U.S. patent application Ser. No. 11/807,872, filed May 29, 2007, the disclosure of which is herein incorporated by reference in its entirety for all purposes. Non-limiting examples of siRNA molecules targeting the PLK-1 gene include those described in U.S. Patent Publication Nos. 20050107316 and 20070265438; and U.S. patent application Ser. No. 12/343,342, filed Dec. 23, 2008, the disclosures of which are herein incorporated by reference in their entirety for all purposes. Non-limiting examples of siRNA molecules targeting the CSN5 gene include those described in U.S. Provisional Application No. 61/045,251, filed Apr. 15, 2008, the disclosure of which is herein incorporated by reference in its entirety for all purposes.

Additional examples of gene sequences associated with tumorigenesis and cell transformation include translocation sequences such as MLL fusion genes, BCR-ABL (Wilda et al., Oncogene, 21:5716 (2002); Scherr et al., Blood, 101:1566 (2003)), TEL-AML1, EWS-FLI1, TLS-FUS, PAX3-FKHR, BCL-2, AML1-ETO, and AML1-MTG8 (Heidenreich et al., Blood, 101:3157 (2003)); overexpressed sequences such as multidrug resistance genes (Nieth et al., FEBS Lett., 545:144 (2003); Wu et al, Cancer Res. 63:1515 (2003)), cyclins (Li et al., Cancer Res., 63:3593 (2003); Zou et al., Genes Dev., 16:2923 (2002)), beta-catenin (Verma et al., Clin Cancer Res., 9:1291 (2003)), telomerase genes (Kosciolek et al., Mol Cancer Ther., 2:209 (2003)), c-MYC, N-MYC, BCL-2, growth factor receptors (e.g., EGFR/ErbB1 (Genbank Accession Nos. NM_005228, NM_201282, NM_201283, and NM_201284; see also, Nagy et al. Exp. Cell Res., 285:39-49 (2003), ErbB2/HER-2 (Genbank Accession Nos. NM_004448 and NM_001005862), ErbB3 (Genbank Accession Nos. NM 001982 and NM_001005915), and ErbB4 (Genbank Accession Nos. NM_005235 and NM_001042599); and mutated sequences such as RAS (reviewed in Tuschl and Borkhardt, Mol. Interventions, 2:158 (2002)). Non-limiting examples of siRNA molecules targeting the EGFR gene include those described in U.S. patent application Ser. No. 11/807,872, filed May 29, 2007, the disclosure of which is herein incorporated by reference in its entirety for all purposes.

Silencing of sequences that encode DNA repair enzymes find use in combination with the administration of chemotherapeutic agents (Collis et al., Cancer Res., 63:1550 (2003)). Genes encoding proteins associated with tumor migration are also target sequences of interest, for example, integrins, selectins, and metalloproteinases. The foregoing examples are not exclusive. Those of skill in the art will understand that any whole or partial gene sequence that facilitates or promotes tumorigenesis or cell transformation, tumor growth, or tumor migration can be included as a template sequence.

Angiogenic genes are able to promote the formation of new vessels. Of particular interest is vascular endothelial growth factor (VEGF) (Reich et al., Mol. Vis., 9:210 (2003)) or VEGFR. siRNA sequences that target VEGFR are set forth in, e.g., GB 2396864; U.S. Patent Publication No. 20040142895; and CA 2456444, the disclosures of which are herein incorporated by reference in their entirety for all purposes.

Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin (see, e.g., U.S. Pat. No. 6,174,861), angiostatin (see, e.g., U.S. Pat. No. 5,639,725), and VEGFR2 (see, e.g., Decaussin et al., J. Pathol., 188: 369-377 (1999)), the disclosures of which are herein incorporated by reference in their entirety for all purposes.

Immunomodulator genes are genes that modulate one or more immune responses. Examples of immunomodulator genes include, without limitation, cytokines such as growth factors (e.g., TGF-α, TGF-□β, EGF, FGF, IGF, NGF, PDGF, CGF, GM-CSF, SCF, etc.), interleukins (e.g., IL-2, IL-4, IL-12 (Hill et al., J. Immunol., 171:691 (2003)), IL-15, IL-18, IL-20, etc.), interferons (e.g., IFN-α, IFN-β, IFN-γ, etc.) and TNF. Fas and Fas ligand genes are also immunomodulator target sequences of interest (Song et al., Nat. Med., 9:347 (2003)). Genes encoding secondary signaling molecules in hematopoietic and lymphoid cells are also included in the present invention, for example, Tec family kinases such as Bruton's tyrosine kinase (Btk) (Heinonen et al., FESS Lett., 527:274 (2002)).

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g., inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, glucagon, G-protein coupled receptor ligands, etc. Templates coding for an expansion of trinucleotide repeats (e.g., CAG repeats) find use in silencing pathogenic sequences in neurodegenerative disorders caused by the expansion of trinucleotide repeats, such as spinobulbular muscular atrophy and Huntington's Disease (Caplen et al., Hum. Mol. Genet., 11:175 (2002)).

In addition to its utility in silencing the expression of any of the above-described genes for therapeutic purposes, the siRNA described herein are also useful in research and development applications as well as diagnostic, prophylactic, prognostic, clinical, and other healthcare applications. As a non-limiting example, the siRNA can be used in target validation studies directed at testing whether a gene of interest has the potential to be a therapeutic target. The siRNA can also be used in target identification studies aimed at discovering genes as potential therapeutic targets.

2. aiRNA

Like siRNA, asymmetrical interfering RNA (aiRNA) can recruit the RNA-induced silencing complex (RISC) and lead to effective silencing of a variety of genes in mammalian cells by mediating sequence-specific cleavage of the target sequence between nucleotide 10 and 11 relative to the 5′ end of the antisense strand (Sun et al., Nat. Biotech., 26:1379-1382 (2008)). Typically, an aiRNA molecule comprises a short RNA duplex having a sense strand and an antisense strand, wherein the duplex contains overhangs at the 3′ and 5′ ends of the antisense strand. The aiRNA is generally asymmetric because the sense strand is shorter on both ends when compared to the complementary antisense strand. In some aspects, aiRNA molecules may be designed, synthesized, and annealed under conditions similar to those used for siRNA molecules. As a non-limiting example, aiRNA sequences may be selected and generated using the methods described above for selecting siRNA sequences.

In another embodiment, aiRNA duplexes of various lengths (e.g., about 10-25, 12-20, 12-19, 12-18, 13-17, or 14-17 base pairs, more typically 12, 13, 14, 15, 16, 17, 18, 19, or 20 base pairs) may be designed with overhangs at the 3′ and 5′ ends of the antisense strand to target an mRNA of interest. In certain instances, the sense strand of the aiRNA molecule is about 10-25, 12-20, 12-19, 12-18, 13-17, or 14-17 nucleotides in length, more typically 12, 13, 14, 15, 16, 17, 18, 19, or 20 nucleotides in length. In certain other instances, the antisense strand of the aiRNA molecule is about 15-60, 15-50, or 15-40 nucleotides in length, more typically about 15-30, 15-25, or 19-25 nucleotides in length, and is preferably about 20-24, 21-22, or 21-23 nucleotides in length.

In some embodiments, the 5′ antisense overhang contains one, two, three, four, or more nontargeting nucleotides (e.g., “AA”, “UU”, “dTdT”, etc.). In other embodiments, the 3′ antisense overhang contains one, two, three, four, or more nontargeting nucleotides (e.g., “AA”, “UU”, “dTdT”, etc.). In certain aspects, the aiRNA molecules described herein may comprise one or more modified nucleotides, e.g., in the double-stranded (duplex) region and/or in the antisense overhangs. As a non-limiting example, aiRNA sequences may comprise one or more of the modified nucleotides described above for siRNA sequences. In a preferred embodiment, the aiRNA molecule comprises 2′OMe nucleotides such as, for example, 2′OMe-guanosine nucleotides, 2′OMe-uridine nucleotides, or mixtures thereof.

In certain embodiments, aiRNA molecules may comprise an antisense strand which corresponds to the antisense strand of an siRNA molecule, e.g., one of the siRNA molecules described herein. In other embodiments, aiRNA molecules may be used to silence the expression of any of the target genes set forth above, such as, e.g., genes associated with viral infection and survival, genes associated with metabolic diseases and disorders, genes associated with tumorigenesis and cell transformation, angiogenic genes, immunomodulator genes such as those associated with inflammatory and autoimmune responses, ligand receptor genes, and genes associated with neurodegenerative disorders.

3. miRNA

Generally, microRNAs (miRNA) are single-stranded RNA molecules of about 21-23 nucleotides in length which regulate gene expression. miRNAs are encoded by genes from whose DNA they are transcribed, but miRNAs are not translated into protein (non-coding RNA); instead, each primary transcript (a pri-miRNA) is processed into a short stem-loop structure called a pre-miRNA and finally into a functional mature miRNA. Mature miRNA molecules are either partially or completely complementary to one or more messenger RNA (mRNA) molecules, and their main function is to downregulate gene expression. The identification of miRNA molecules is described, e.g., in Lagos-Quintana at al., Science, 294:853-858; Lau et al., Science, 294:858-862; and Lee et al., Science, 294:862-864.

The genes encoding miRNA are much longer than the processed mature miRNA molecule. miRNA are first transcribed as primary transcripts or pri-miRNA with a cap and poly-A tail and processed to short, ˜70-nucleotide stem-loop structures known as pre-miRNA in the cell nucleus. This processing is performed in animals by a protein complex known as the Microprocessor complex, consisting of the nuclease Drosha and the double-stranded RNA binding protein Pasha (Denli et al., Nature, 432:231-235 (2004)). These pre-miRNA are then processed to mature miRNA in the cytoplasm by interaction with the endonuclease Dicer, which also initiates the formation of the RNA-induced silencing complex (RISC) (Bernstein et al., Nature, 409:363-366 (2001). Either the sense strand or antisense strand of DNA can function as templates to give rise to miRNA.

When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end (Preall et al., Curr. Biol., 16:530-535 (2006)). The remaining strand, known as the anti-guide or passenger strand, is degraded as a RISC complex substrate (Gregory et al., Cell, 123:631-640 (2005)). After integration into the active RISC complex, miRNAs base pair with their complementary mRNA molecules and induce target mRNA degradation and/or translational silencing.

Mammalian miRNA molecules are usually complementary to a site in the 3′ UTR of the target mRNA sequence. In certain instances, the annealing of the miRNA to the target mRNA inhibits protein translation by blocking the protein translation machinery. In certain other instances, the annealing of the miRNA to the target mRNA facilitates the cleavage and degradation of the target mRNA through a process similar to RNA interference (RNAi). miRNA may also target methylation of genomic sites which correspond to targeted mRNA. Generally, miRNA function in association with a complement of proteins collectively termed the miRNP.

In certain aspects, the miRNA molecules described herein are about 15-100, 15-90, 15-80, 15-75, 15-70, 15-60, 15-50, or 15-40 nucleotides in length, more typically about 15-30, 15-25, or 19-25 nucleotides in length, and are preferably about 20-24, 21-22, or 21-23 nucleotides in length. In certain other aspects, miRNA molecules may comprise one or more modified nucleotides. As a non-limiting example, miRNA sequences may comprise one or more of the modified nucleotides described above for siRNA sequences. In a preferred embodiment, the miRNA molecule comprises 2′OMe nucleotides such as, for example, 2′OMe-guanosine nucleotides, 2′OMe-uridine nucleotides, or mixtures thereof.

In some embodiments, miRNA molecules may be used to silence the expression of any of the target genes set forth above, such as, e.g., genes associated with viral infection and survival, genes associated with metabolic diseases and disorders, genes associated with tumorigenesis and cell transformation, angiogenic genes, immunomodulator genes such as those associated with inflammatory and autoimmune responses, ligand receptor genes, and genes associated with neurodegenerative disorders.

In other embodiments, one or more agents that block the activity of a miRNA targeting an mRNA of interest are administered using a lipid particle of the invention (e.g., a nucleic acid-lipid particle). Examples of blocking agents include, but are not limited to, steric blocking oligonucleotides, locked nucleic acid oligonucleotides, and Morpholino oligonucleotides. Such blocking agents may bind directly to the miRNA or to the miRNA binding site on the target mRNA.

### 4. Antisense Oligonucleotides

In one embodiment, the nucleic acid is an antisense oligonucleotide directed to a target gene or sequence of interest. The terms “antisense oligonucleotide” or “antisense” include oligonucleotides that are complementary to a targeted polynucleotide sequence. Antisense oligonucleotides are single strands of DNA or RNA that are complementary to a chosen sequence. Antisense RNA oligonucleotides prevent the translation of complementary RNA strands by binding to the RNA. Antisense DNA oligonucleotides can be used to target a specific, complementary (coding or non-coding) RNA. If binding occurs, this DNA/RNA hybrid can be degraded by the enzyme RNase H. In a particular embodiment, antisense oligonucleotides comprise from about 10 to about 60 nucleotides, more preferably from about 15 to about 30 nucleotides. The term also encompasses antisense oligonucleotides that may not be exactly complementary to the desired target gene. Thus, the invention can be utilized in instances where non-target specific-activities are found with antisense, or where an antisense sequence containing one or more mismatches with the target sequence is the most preferred for a particular use.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (see, U.S. Pat. Nos. 5,739,119 and 5,759,829). Furthermore, examples of antisense inhibition have been demonstrated with the nuclear protein cyclin, the multiple drug resistance gene (MDR1), ICAM-1, E-selectin, STK-1, striatal GABAA receptor, and human EGF (see, Jaskulski et al., Science, 240:1544-6 (1988); Vasanthakumar et al., Cancer Commun., 1:225-32 (1989); Penis et al., Brain Res Mal Brain Res., 15; 57:310-20 (1998); and U.S. Pat. Nos. 5,801,154; 5,789,573; 5,718,709 and 5,610,288). Moreover, antisense constructs have also been described that inhibit and can be used to treat a variety of abnormal cellular proliferations, e.g., cancer (see, U.S. Pat. Nos. 5,747,470; 5,591,317; and 5,783,683). The disclosures of these references are herein incorporated by reference in their entirety for all purposes.

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res., 25:3389-402 (1997)).

### 5. Ribozymes

According to another embodiment of the invention, nucleic acid-lipid particles are associated with ribozymes. Ribozymes are RNA-protein complexes having specific catalytic domains that possess endonuclease activity (see, Kim et al., Proc. Natl. Acad Sci. USA., 84:8788-92 (1987); and Forster et al., Cell, 49:211-20 (1987)). For example, a large number of ribozymes accelerate phosphoester transfer reactions with a high degree of specificity, often cleaving only one of several phosphoesters in an oligonucleotide substrate (see, Cech et al., Cell, 27:487-96 (1981); Michel et al., J. Mol. Biol., 216:585-610 (1990); Reinhold-Hurek et al., Nature, 357:173-6 (1992)). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNA molecules are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, hepatitis δ virus, group I intron or RNaseP RNA (in association with an RNA guide sequence), or Neurospora VS RNA motif, for example. Specific examples of hammerhead motifs are described in, e.g., Rossi et al., Nucleic Acids Res., 20:4559-65 (1992). Examples of hairpin motifs are described in, e.g., EP 0360257, Hampel et al., Biochemistry, 28:4929-33 (1989); Hampel et al., Nucleic Acids Res., 18:299-304 (1990); and U.S. Pat. No. 5,631,359. An example of the hepatitis δ virus motif is described in, e.g., Perrotta et al., Biochemistry, 31:11843-52 (1992). An example of the RNaseP motif is described in, e.g., Guerrier-Takada et al., Cell, 35:849-57 (1983). Examples of the Neurospora VS RNA ribozyme motif is described in, e.g., Saville et al., Cell, 61:685-96 (1990); Saville et al., Proc. Natl. Acad. Sci. USA, 88:8826-30 (1991); Collins et al., Biochemistry, 32:2795-9 (1993). An example of the Group I intron is described in, e.g., U.S. Pat. No. 4,987,071. Important characteristics of enzymatic nucleic acid molecules used according to the invention are that they have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein. The disclosures of these references are herein incorporated by reference in their entirety for all purposes.

Methods of producing a ribozyme targeted to any polynucleotide sequence are known in the art. Ribozymes may be designed as described in, e.g., PCT Publication Nos. WO 93/23569 and WO 94/02595, and synthesized to be tested in vitro and/or in vivo as described therein. The disclosures of these PCT publications are herein incorporated by reference in their entirety for all purposes.

Ribozyme activity can be optimized by altering the length of the ribozyme binding arms or chemically synthesizing ribozymes with modifications that prevent their degradation by serum ribonucleases (see, e.g., PCT Publication Nos. WO 92/07065, WO 93/15187, WO 91/03162, and WO 94/13688; EP 92110298.4; and U.S. Pat. No. 5,334,711, which describe various chemical modifications that can be made to the sugar moieties of enzymatic RNA molecules, the disclosures of which are each herein incorporated by reference in their entirety for all purposes), modifications which enhance their efficacy in cells, and removal of stem II bases to shorten RNA synthesis times and reduce chemical requirements.

### 6. Immunostimulatory Oligonucleotides

Nucleic acids associated with lipid particles of the present invention may be immunostimulatory, including immunostimulatory oligonucleotides (ISS; single- or double-stranded) capable of inducing an immune response when administered to a subject, which may be a mammal such as a human. ISS include, e.g., certain palindromes leading to hairpin secondary structures (see, Yamamoto et al., J. Immunol., 148:4072-6 (1992)), or CpG motifs, as well as other known ISS features (such as multi-G domains; see; PCT Publication No. WO 96/11266, the disclosure of which is herein incorporated by reference in its entirety for all purposes).

Immunostimulatory nucleic acids are considered to be non-sequence specific when it is not required that they specifically bind to and reduce the expression of a target sequence in order to provoke an immune response. Thus, certain immunostimulatory nucleic acids may comprise a sequence corresponding to a region of a naturally-occurring gene or mRNA, but they may still be considered non-sequence specific immunostimulatory nucleic acids.

In one embodiment, the immunostimulatory nucleic acid or oligonucleotide comprises at least one CpG dinucleotide. The oligonucleotide or CpG dinucleotide may be unmethylated or methylated. In another embodiment, the immunostimulatory nucleic acid comprises at least one CpG dinucleotide having a methylated cytosine. In one embodiment, the nucleic acid comprises a single CpG dinucleotide, wherein the cytosine in the CpG dinucleotide is methylated. In an alternative embodiment, the nucleic acid comprises at least two CpG dinucleotides, wherein at least one cytosine in the CpG dinucleotides is methylated. In a further embodiment, each cytosine in the CpG dinucleotides present in the sequence is methylated. In another embodiment, the nucleic acid comprises a plurality of CpG dinucleotides, wherein at least one of the CpG dinucleotides comprises a methylated cytosine. Examples of immunostimulatory oligonucleotides suitable for use in the compositions and methods of the present invention are described in PCT Application No. PCT/US08/88676, filed Dec. 31, 2008, PCT Publication Nos. WO 02/069369 and WO 01/15726, U.S. Pat. No. 6,406,705, and Raney et al., J. Pharm. Exper. Ther., 298:1185-92 (2001), the disclosures of which are each herein incorporated by reference in their entirety for all purposes. In certain embodiments, the oligonucleotides used in the compositions and methods of the invention have a phosphodiester (“PO”) backbone or a phosphorothioate (“PS”) backbone, and/or at least one methylated cytosine residue in a CpG motif.

### B. Other Active Agents

In certain embodiments, the active agent associated with the lipid nanoparticles of the invention may comprise one or more therapeutic proteins, polypeptides, or small organic molecules or compounds. Non-limiting examples of such therapeutically effective agents or drugs include oncology drugs (e.g., chemotherapy drugs, hormonal therapeutic agents, immunotherapeutic agents, radiotherapeutic agents, etc.), lipid-lowering agents, anti-viral drugs, anti-inflammatory compounds, antidepressants, stimulants, analgesics, antibiotics, birth control medication, antipyretics, vasodilators, anti-angiogenics, cytovascular agents, signal transduction inhibitors, cardiovascular drugs such as anti-arrhythmic agents, hormones, vasoconstrictors, and steroids. These active agents may be administered alone in the lipid particles of the invention, or in combination (e.g., co-administered) with lipid particles of the invention comprising nucleic acid such as interfering RNA.

Non-limiting examples of chemotherapy drugs include platinum-based drugs (e.g., oxaliplatin, cisplatin, carboplatin, spiroplatin, iproplatin, satraplatin, etc.), alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil (5-FU), azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel (taxol), docetaxel, etc.), topoisomerase inhibitors (e.g., irinotecan (CPT-11; Camptosar), topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), tyrosine kinase inhibitors (e.g., gefitinib (Iressa®), sunitinib (Sutent®; SU11248), erlotinib (Tarceva®; OSI-1774), lapatinib (GW572016; GW2016), canertinib (CI 1033), semaxinib (SU5416), vatalanib (PTK787/ZK222584), sorafenib (BAY 43-9006), imatinib (Gleevec®; ST1571), dasatinib (BMS-354825), leflunomide (SU101), vandetanib (Zactima™; ZD6474), etc.), pharmaceutically acceptable salts thereof, stereoisomers thereof, derivatives thereof, analogs thereof, and combinations thereof.

Examples of conventional hormonal therapeutic agents include, without limitation, steroids (e.g., dexamethasone), finasteride, aromatase inhibitors, tamoxifen, and goserelin as well as other gonadotropin-releasing hormone agonists (GnRH).

Examples of conventional immunotherapeutic agents include, but are not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), and radioimmunotherapy (e.g., anti-CD20 monoclonal antibody conjugated to ¹¹¹In, ⁹⁰Y, or ¹³¹I etc.).

Examples of conventional radiotherapeutic agents include, but are not limited to, radionuclides such as ⁴⁷Sc, ⁶⁴Cu, ⁶⁷Cu, ³⁷Sr, ⁸⁶Y, ³⁷Y, ⁹⁰Y, ¹⁰⁵Rh, ¹¹¹Ag, ¹¹¹In, ^{117m}Sn, ¹⁴⁹Pm ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

Additional oncology drugs that may be used according to the invention include, but are not limited to, alkeran, allopurinol, altretamine, amifostine, anastrozole, araC, arsenic trioxide, bexarotene, biCNU, carmustine, CCNU, celecoxib, cladribine, cyclosporin A, cytosine arabinoside, cytoxan, dexrazoxane, DTIC, estramustine, exemestane, FK506, gemtuzumab-ozogamicin, hydrea, hydroxyurea, idarubicin, interferon, letrozole, leustatin, leuprolide, litertinoin, megastrol, L-PAM, mesna, methoxsalen, mithramycin, nitrogen mustard, pamidronate, Pegademase, pentostatin, porfimer sodium, prednisone, rituxan, streptozocin, STI-571, taxotere, temozolamide, VM-26, toremifene, tretinoin, ATRA, valrubicin, and velban. Other examples of oncology drugs that may be used according to the invention are ellipticin and ellipticin analogs or derivatives, epothilones, intracellular kinase inhibitors, and camptothecins.

Non-limiting examples of lipid-lowering agents for treating a lipid disease or disorder associated with elevated triglycerides, cholesterol, and/or glucose include statins, fibrates, ezetimibe, thiazolidinediones, niacin, beta-blockers, nitroglycerin, calcium antagonists, fish oil, and mixtures thereof.

Examples of anti-viral drugs include, but are not limited to, abacavir, aciclovir, acyclovir, adefovir, amantadine, amprenavir, arbidol, atazanavir, atripla, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscarnet, fosfonet, fusion inhibitors, ganciclovir, ibacitabine, imunovir, idoxuridine, imiquimod, indinavir, inosine, integrase inhibitors, interferon type Ill (e.g., IFN-A molecules such as IFN-λ1, IFN-λ2, and IFN-λ3), interferon type II (e.g., IFN-γ), interferon type I (e.g., IFN-α such as PEGylated IFN-α, IFN-β, IFN-κ, IFN-δ, IFN-ε, IFN-τ, IFN-ω, and IFN-ξ, interferon, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, pharmaceutically acceptable salts thereof, stereoisomers thereof, derivatives thereof, analogs thereof, and mixtures thereof.

Additional drugs include zotarolimus, sirolimus, rapamycin, everolimus, biolimus, myolimus, novolimus, temsirolimus, deforolimus, merilimus, tacrolimus, pimecrolimus, ridaforolimus pharmaceutically acceptable salts thereof, stereoisomers thereof, derivatives thereof, analogs thereof, and mixtures thereof.

### Pharmaceutical Compositions

Lipid nanoparticles may be formulated in whole or in part as pharmaceutical compositions. Pharmaceutical compositions may include one or more nanoparticles. For example, a pharmaceutical composition may include one or more nanoparticles including one or more different therapeutic and/or prophylactic agents. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a lipid nanoparticle. An excipient or accessory ingredient may be incompatible with a component of a lipid nanoparticle if its combination with the component may result in any undesirable biological effect or otherwise deleterious effect.

In some embodiments, one or more excipients or accessory ingredients may make up greater than 50% of the total mass or volume of a pharmaceutical composition including a lipid nanoparticle. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles.

In certain embodiments, the lipid nanoparticles and/or pharmaceutical compositions of the invention are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.)

In certain embodiments, the pharmaceutical composition of the invention comprises a lipid nanoparticle disclosed herein and a pharmaceutically acceptable carrier selected from one or more of Tris, an acetate (e.g., sodium acetate), an citrate (e.g., sodium citrate), saline, PBS, and sucrose. In certain embodiments, the pharmaceutical composition of the disclosure has a pH value between about 7 and 8 (e.g., 6.8 6.9, 7.0, 7.1, 7.2, 7.3, 7.4, 7.5, 7.6, 7.7, 7.8, 7.9 or 8.0, or between 7.5 and 8 or between 7 and 7.8). For example, a pharmaceutical composition of the disclosure comprises a nanoparticle composition disclosed herein, Tris, saline and sucrose, and has a pH of about 7.5-8, which is suitable for storage and/or shipment at, for example, about −20° C. For example, a pharmaceutical composition of the disclosure comprises a lipid nanoparticle disclosed herein and PBS and has a pH of about 7-7.8, suitable for storage and/or shipment at, for example, about 4° C. or lower. “Stability,” “stabilized,” and “stable” in the context of the present disclosure refers to the resistance of nanoparticle compositions and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

In a preferred embodiment, the lipid nanoparticles of the invention are formulated with one or more disaccharides, or disaccharide containing molecules, such as sucrose, lactose, maltose, trehalose, maltitol or lactitol in any one or more of the buffers described herein, including Tris HCl, Tris Acetate, TT/AA. As shown in the Examples below, lipid nanoparticles formulated with sucrose can be reconstituted after thawing without aggregation.

Lipid nanoparticles and/or pharmaceutical compositions including one or more lipid nanoparticles may be administered to any patient or subject, including those patients or subjects that may benefit from a therapeutic effect provided by the delivery of a therapeutic and/or prophylactic to one or more particular cells, tissues, organs, or systems or groups thereof, such as the renal system. Although the descriptions provided herein of lipid nanoparticles and pharmaceutical compositions including lipid nanoparticles are principally directed to compositions which are suitable for administration to humans, it will be understood by the skilled artisan that such compositions are generally suitable for administration to any other mammal.

Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more lipid nanoparticles may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage such as, for example, one-half or one-third of such a dosage.

Pharmaceutical compositions may be prepared in a variety of forms suitable for a variety of routes and methods of administration. For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutic and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay, silicates), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Methods of Treating Diseases and Disorders

Lipid nanoparticles may be useful for treating a disease, disorder, or condition. In particular, such compositions may be useful in treating a disease, disorder, or condition characterized by missing or aberrant protein or polypeptide activity. For example, a nanoparticle composition comprising an active, such as mRNA encoding a missing or aberrant polypeptide, may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment of acute diseases, disorders, or conditions such as sepsis, stroke, and myocardial infarction. A therapeutic and/or prophylactic included in a nanoparticle composition may also be capable of altering the rate of transcription of a given species, thereby affecting gene expression.

Diseases, disorders, and/or conditions characterized by dysfunctional or aberrant protein or polypeptide activity for which a composition may be administered include, but are not limited to, rare diseases, infectious diseases (as both vaccines and therapeutics), cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis.

The present disclosure provides a method for treating such diseases, disorders, and/or conditions in a subject by administering a lipid nanoparticle of the invention including an RNA, wherein the RNA may be an mRNA encoding a polypeptide that antagonizes or otherwise overcomes an aberrant protein activity present in the cell of the subject.

The disclosure provides methods involving administering lipid nanoparticles including one or more therapeutic and/or prophylactic agents and pharmaceutical compositions including the same. The terms therapeutic and prophylactic can be used interchangeably herein with respect to features and embodiments of the present disclosure. Therapeutic compositions, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a lipid nanoparticle or pharmaceutical composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more therapeutic and/or prophylactic agents employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A lipid nanoparticle including one or more therapeutic and/or prophylactic agents may be administered by any route. In some embodiments, compositions, including prophylactic, diagnostic, or imaging compositions including one or more lipid nanoparticles described herein, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, intraocular, subretinal, intravitreal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, intraocularly, subretinally, intravitreally, or by inhalation. In general, the most appropriate route of administration will depend upon a variety of factors including the nature of the nanoparticle composition including one or more therapeutic and/or prophylactics (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic and/or prophylactic agent (e.g., an mRNA) in a given dose, where a dose of 1 mg/kg (mpk) provides 1 mg of a therapeutic and/or prophylactic per 1 kg of subject body weight. In some embodiments, a dose of about 0.001 mg/kg to about 10 mg/kg of a therapeutic and/or prophylactic (e.g., mRNA) of a nanoparticle composition may be administered. In other embodiments, a dose of about 0.005 mg/kg to about 2.5 mg/kg of a therapeutic and/or prophylactic may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Lipid nanoparticles of the invention including one or more therapeutic and/or prophylactic agents may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more lipid nanoparticles including one or more different therapeutic and/or prophylactic agents may be administered in combination. Lipid nanoparticles can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of lipid nanoparticles, compositions, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

### EXAMPLES

### Example 1—Generation of Lipid Nanoparticles

Nanoparticles can be made with mixing processes such as microfluidics and T-junction mixing of two fluid streams, one of which contains the therapeutic and/or prophylactic and the other has the lipid components. Lipid compositions are prepared by combining a cationic lipid (such as DODAC, DODAP or Dlim-MC3-DMA), a phospholipid (such as DOPE or DSPC) a PEG lipid (such as 1,2-dimyristoyl-rac-glycero-3-methoxypolyethylene glycol-2000, also known as DMG-PEG) and a structural lipid (such as cholesterol or a corticosteroid (such as prednisolone, dexamethasone, prednisone, and hydrocortisone), or a combination thereof) at concentrations of about 50 mM in ethanol. Unless specified otherwise, the nanoparticle referred to as “MIPS-LNP” herein includes any one of the lipid nanoparticles 1 to 4 referred to below. Solutions should be refrigerated for storage at, for example, 4° C. or frozen for storage at, for example −20° C. Lipids are combined to yield desired molar ratios and diluted with ethanol to a final lipid concentration of between about 5.5 mM and about 50 mM. Nanoparticle compositions including a therapeutic and/or prophylactic and a lipid component are prepared by combining the lipid solution with a solution including the therapeutic and/or prophylactic at lipid components to therapeutic and/or prophylactic wt:wt ratios between about 5:1 and about 50:1. The lipid solution is mixed with the nucleic acid solution, for example using a microfluidic or T-junction based system, at flow rates between about 0.5 ml/min and about 8 ml/min to produce a lipid nanoparticle suspension with a water to ethanol ratio between about 1:1 and about 4:1 preferably 3:1. Where the NP ratio (nitrogen to phosphate) is maintained between 4-7. The solution can be immediately diluted with buffer of choice or buffer exchanged as it is described below.

Examples of lipid nanoparticles that have been generated include:

### Lipid Nanoparticle 1

| | | mol % of the total lipid |
|---|---|---|
| Lipid class | Lipid Type | present in the nanoparticle |
| cationic and/or ionisable | Dlin-MC3-DMA | 50% |
| lipid | | |
| phospholipid | DSPC | 10% |
| structural lipid | Cholesterol | 39.85%, |
| PEGylated lipid | DMG-PEG | 0.15% |

### Lipid Nanoparticle 2

| | | mol % of the total lipid |
|---|---|---|
| Lipid class | Lipid Type | present in the nanoparticle |
| cationic and/or ionisable | Dlin-MC3-DMA | 50% |
| lipid | | |
| phospholipid | DSPC | 10% |
| structural lipid | Cholesterol | 39.85% |
| PEGylated lipid | DSPE-PEG | 0.15% |

### Lipid Nanoparticle 3

| | | mol % of the total lipid |
|---|---|---|
| Lipid class | Lipid Type | present in the nanoparticle |
| cationic and/or ionisable | DODAP | 50% |
| lipid | | |
| phospholipid | DSPC | 10% |
| structural lipid | Cholesterol | 39.85% |
| PEGylated lipid | DSPE-PEG | 0.15% |

Lipid nanoparticle 4

| | | mol % of the total lipid |
|---|---|---|
| Lipid class | Lipid Type | present in the nanoparticle |
| cationic and/or ionisable | DODAP | 50% |
| lipid | | |
| phospholipid | DSPC | 10% |
| structural lipid | Cholesterol | 39.85%, |
| PEGylated lipid | DMG-PEG | 0.15% |

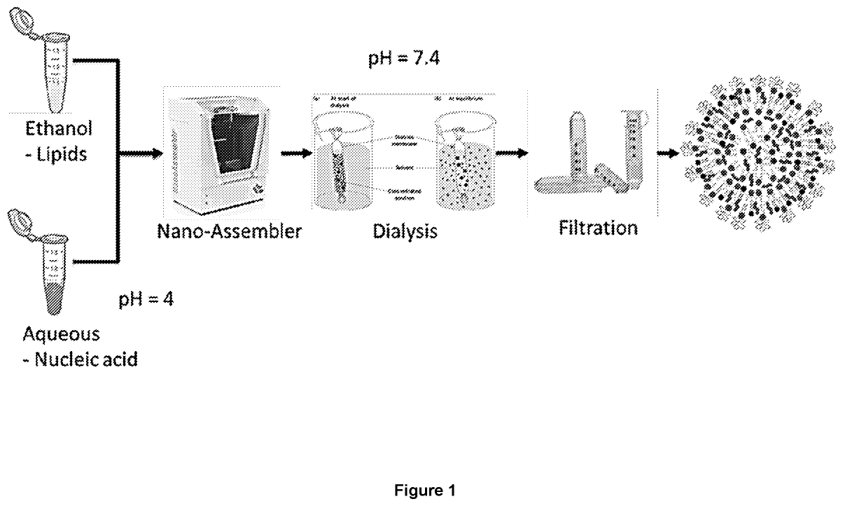

For nanoparticle compositions including an RNA, solutions of the RNA at concentrations of 0.133-0.6 mg/ml in deionized water are diluted in 25 mM sodium acetate buffer at a pH between 3 and 4.5 to form a stock solution (FIG. 1). After nanoparticle formulation, nanoparticle compositions can be processed by dialysis or tangential flow filtration to remove ethanol and achieve buffer exchange. For example, formulations are dialyzed twice against phosphate buffered saline (PBS), pH 7.4, at volumes 200 times that of the primary product using Slide-A-Lyzer cassettes (Thermo Fisher Scientific Inc., Rockford, Ill.) with a molecular weight cutoff of 10 kD. The first dialysis is carried out at room temperature for 3 hours. The formulations are then dialyzed overnight at 4° C. The resulting nanoparticle suspension is filtered through 0.4 or 0.22 μm sterile filters into glass vials and sealed. Nanoparticle composition solutions of 0.01 mg/ml to 0.50 mg/ml are generally obtained. In some embodiments, Tangential flow filtration (TFF), from Repligen, was used instead of the dialysis cassettes. Additionally, Tris-Sucrose, pH 7.4, solution was used to allow some of the formulation to be frozen between −10 to −80° C.

The method described above induces nano-precipitation and particle formation using NanoAssemblr microfluidics. Alternative processes including, but not limited to, T-junction and direct injection, may be used to achieve the same nano-precipitation. Higher or lower concentration of mRNA stock, and encapsulating lipid, can also be used during the nano-precipitation.

LNPs are generally formulated such that after intravenous injection they are passively delivered to the liver. An LNP targeting siRNA to the liver (OnPattro, Alnylam Pharmaceuticals) was approved for human use by the FDA in 2018. The inventors refer to this type of product as a conventional LNP.

### Example 2—Structural Characterisation of Lipid Nanoparticles

The lipid nanoparticle size, polydispersity index (PDI) and the zeta potential of the nanoparticle compositions were determined using Zetasizer (Malvern instruments). In brief, dialysed particles were placed disposable cuvette (e.g. zeta-cell (Malvern) or UV cuvettes). The cuvette was placed inside the Zetasizer and measurement was recorded. Calculation of size were recoded with Zetasizer software 12.0.

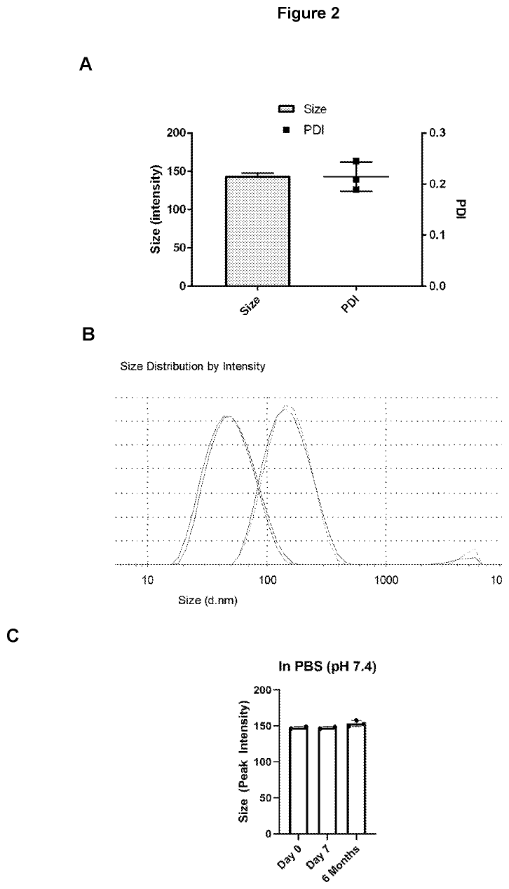

The MIPS-LNP can be produced by reducing the molar ratio of the PEGylated lipid content, below the 0.5 mole % level used in conventional LNP compositions. Commonly, the conventional LNP is formed at 1.5 mole % of PEGylated lipid. The inventors observed that decreasing PEGylated lipid content level of the LNP resulted in larger particle sizes greater than 100 nm (FIG. 2A). In contrast, conventional LNP formulations generate particles of less than 100 nm in size but both MIPs-LNP and conventional LNPs maintain a similar size distribution profile (FIG. 2B). Furthermore, the inventors have shown that MIPS-LNPs 0.15 mol % of PEGylated lipid are stable for at least 6 months at 4° C. with reduced PEGylated mole % level (FIG. 2C). The overall formulation of MIPS-LNPs provides an unusually highly negative zeta potential (data not shown), which will be beneficial to enhance access to immune organs such as the spleen.

Biophysical characterisation described herein was performed on lipid nanoparticles 3 or 4 referred to above.

### Example 3—Functional Characterisation of Lipid Nanoparticles

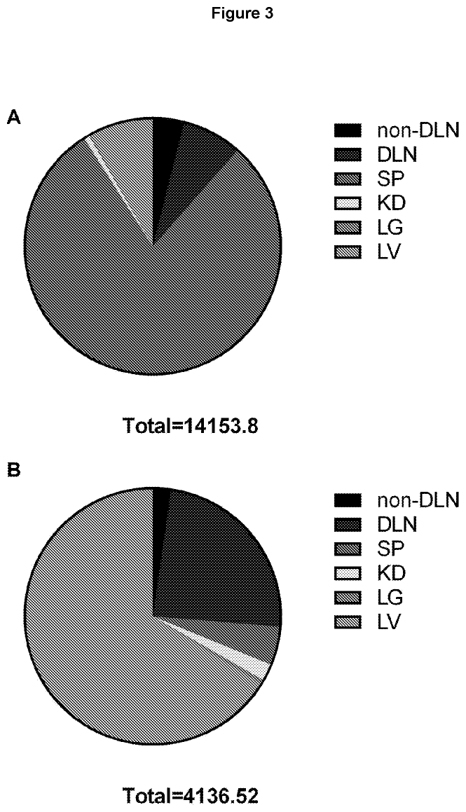

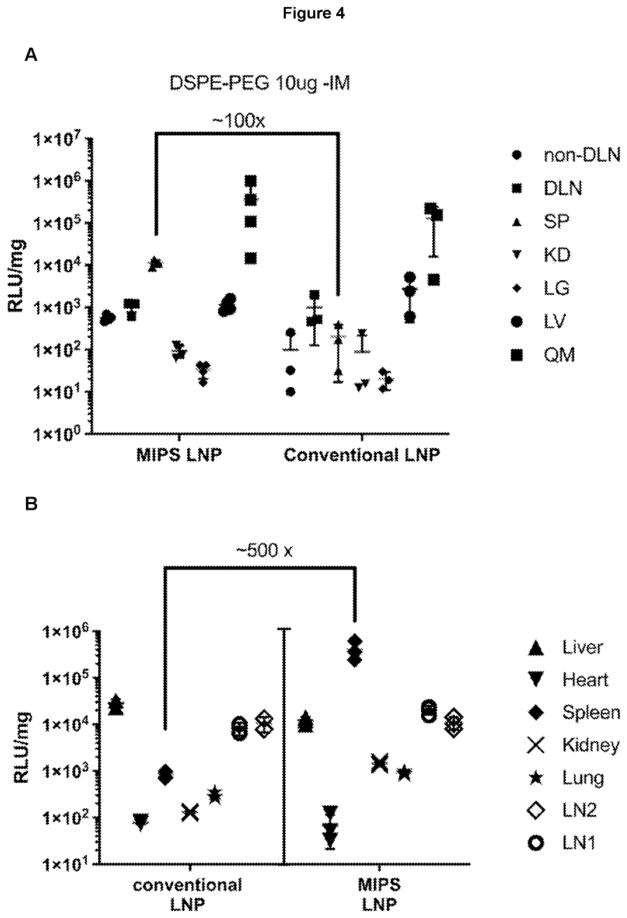

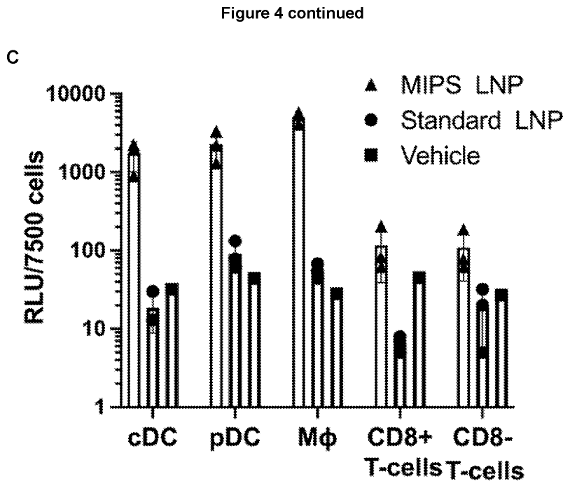

Intramuscular injection (IM) is a common method of administration used for vaccination, particularly for viral and bacterial diseases. Using DODAP as an ionisable lipid (e.g. lipid nanoparticles 3 or 4 referred to above), the inventors demonstrate that after IM injection >75% of the gene expression outside of muscle tissue induced by MIPS-LNP is in the spleen compared with ˜10% induced by conventional LNP (FIG. 3). The delivery of mRNA by MIPS-LNP also surprisingly results in ˜100× higher levels of the reporter gene, nanoluciferase, in the spleen when compared to conventional small LNPs (FIG. 4A). 10 μg nanoluciferase mRNA dose was used in the study. Furthermore, MIPS-LNP is shown to gain access to the lymph nodes that are not immediately downstream of lymph flow from the injection site (known as non-draining lymph nodes).

The inventors have shown that larger average sizes of LNP, lead to better uptake by antigen presenting cells, i.e. the immune cells that orchestrate vaccine responses. Common sizes for current LNPs are in the range 70-100 nm. MIPS-LNPs are designed to be larger than 100 nm in size. Particularly larger than 125 nm in diameter (typically 140-160 nm). Although larger particles can add further selective passive delivery to the spleen (data not shown).

Intravenous (IV) injection is an alternate method of administration that can be used for treatment of hospitalised patients—e.g. cancer vaccination. The inventors have demonstrated that delivery of mRNA by MIPS-LNP (very large LNP) via IV injection using 10 μg nanoluciferase mRNA dose results in ˜500× higher levels of the reporter gene, nanoluciferase, in the spleen (highlighted by a box) when compared to conventional small LNPs (FIG. 4B). Furthermore, MIPS-LNPs demonstrated substantially increased gene delivery through reporter gene expression in a variety of sorted splenocytes compared to conventional LNPs (FIG. 4C). The MIPS-LNP also has the advantage of rerouting the formulation out of the liver (the primary target for use of conventional LNP formulations that were designed for siRNA delivery to the liver). For vaccines, lowering gene expression in the liver can be viewed as beneficial—i.e. reducing the side effects by reducing the “off-target” region. To analyse gene expression in those studies, tissues were collected 16-24 hours after injection, tissues weighed, 1 ml of GloLysis buffer was added and homogenised using M-tubes (Miltenyl). The tubes were centrifuged (10000 g×1 min) and supernatent collected. The analysis of Nanoluciferase gene expression was performed as suggested by NanoGlo assay (Promega). Gene expression levels were quantified on a plate-reader (Perkin elmer) using luciferase setting. Gene expression per g of tissue was calculated and plotted.

Gene expression in splenocytes was determined approximately 16 hours after injection with nanoluciferase (10 ug), Splenocytes were isolated by collagenase digestion (2 mg/ml) and were first labelled with CD8, CD11C, F4/80, mPDCA-1 and the cells were separated using FACS. Separated population were then analysed for luciferase expression (i.e. specific uptake and expression per cell population) was analysed using NanoGlo assay as described above.

In this example MIPS-LNP has a 2.3× lower delivery to the liver compared to conventional LNP (Table 1).

TABLE 1
Nanoluciferase expression in the liver
following intravenous injection.

| | Conventional | MIPS-LNP |
|---|---|---|
| 1 | 23598 | 10000 |
| 2 | 32770 | 12367 |
| 3 | | 14357 |
| Avg | 28184 | 12242 |

### Example 4—Cellular Immune Response to Lipid Nanoparticle Delivery of Antigenic Molecule

To determine the level of immune response, antigen-specific killing assay using ovalbumine antigen was used. C57BL mice (age 8-24 weeks) were vaccinated intravenously.

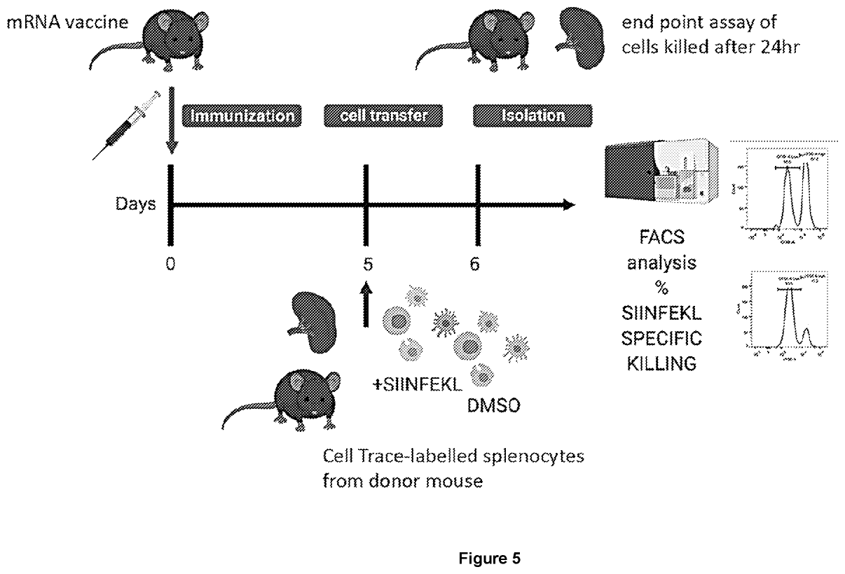

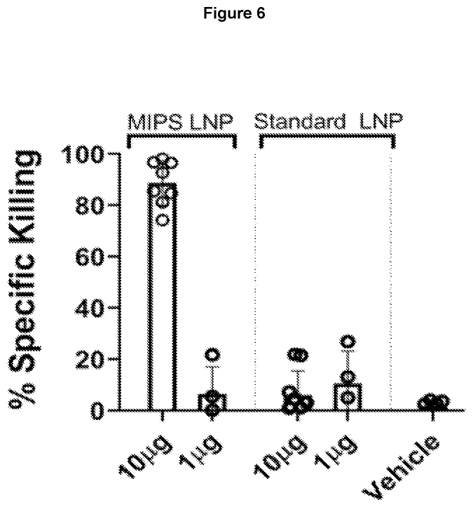

The cellular immune responses to antigenic molecule delivery by MIPS-LNPs the inventors used an in vivo mRNA vaccine model. Mice were immunised with a MIPS-LNP comprising an antigenic ovalbumin mRNA construct on day 0. Cell Trace-labelled SIINFEKL splenocytes were transferred into the immunised mice from donor mice on day 5 and splenocytes from the vaccinated mice were isolated and analysed after 24 hrs (FIG. 5). The level of killing in SIINFEKL pulsed cells (antigen-carrying cell) was compared against cell control (no SIINFEKL). MIPS-LNPs induced an enhanced target-specific cytotoxic T cell-killing against ovalbumin epitopes (FIG. 6). MIPS-LNPs induced almost complete killing even when a small dose of mRNA is delivered (10 ug) to the mice. In comparison, conventional LNP did not produce effective vaccination at the same dose of mRNA. The different percentage between antigen-lacking and antigen containing (killed) compared to pre-injection was measured using flow cytometer and plotted as percentage.

### Example 5—Physical Stability of MIPS-LNPs

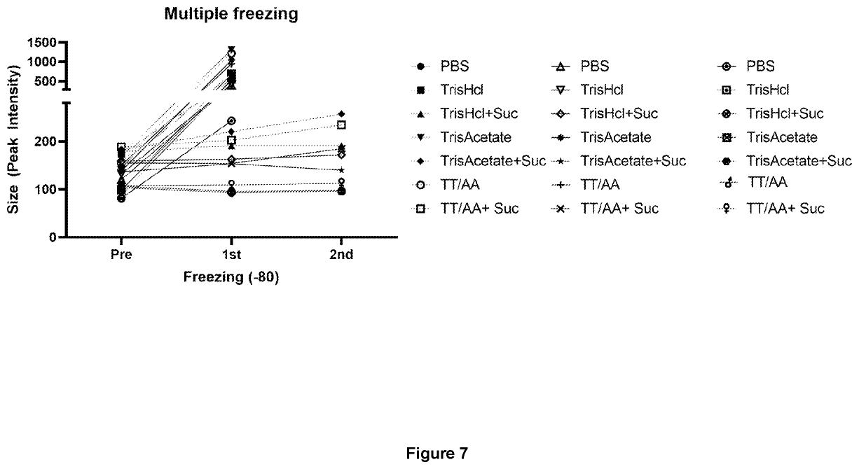

To establish that a pharmaceutical product is feasible using the MIPS formulation the inventors tested whether the formulation can be ‘freeze-thawed’, enabling storage in a freezer for transport prior to use. The product (lipid nanoparticle 1 above) was formulated and dialysed in a variety of buffers as described above and froze multiple times (FIG. 7). All formulations with the addition of sucrose as a cryoprotectant demonstrated particle reconstitution after thawing without significant aggregation. The MIPS-LNP formulation is therefore shown to be stable for transportation and demonstrates its feasible use as a pharmaceutical product.

### Example 6—the Effect of PEGlylated Lipid Content on LNP Delivery

To establish the effects of PEGlyated lipid concentration on LNP delivery, the inventors prepared two formulations containing DLin-MC3-DMA (50 mole %), distearoylphosphatidylcholine (DSPC) (10 mole %), PEGylated lipid (0.15 mole % or 1.5 mole %), cholesterol (remainder of the lipid content—i.e 39.85 mole % or 38.5 mole % respectively). DSPE-PEG or DMG-PEG were used as the PEGylated lipid. Nanoluciferase mRNA was packaged into the LNP formulations (“0.15 mole %” or “1.5 mole %”) and injected intramuscular (IM) or intravenously (IV) into BalbC mice (aged 8-24 weeks). 14-18 hours later, the organs were isolated and weighed. The organs were homogenised using M-tubes (Miltenyi Biotec) and using Glo-lysis buffer (Promega). Nanoluciferase activity as (RLU) were measured using Nanoluciferase lysis kit (Nano-Glo® Luciferase Assay System, Promega) following the manufacturer's instructions.

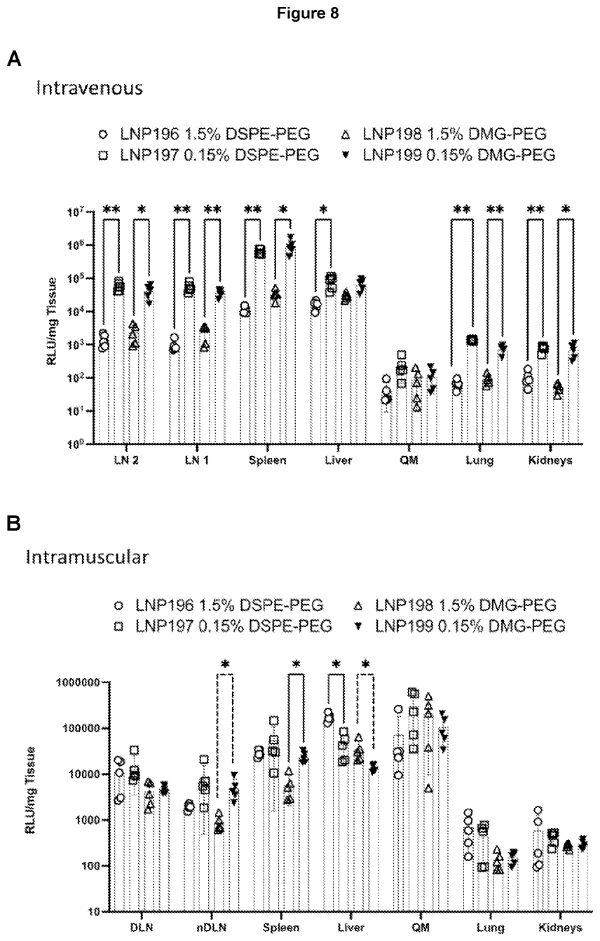

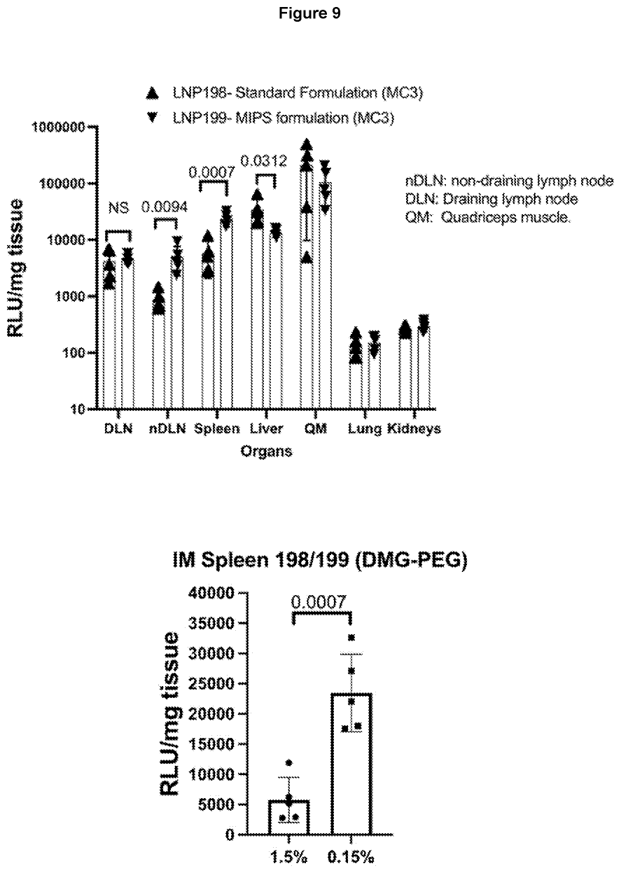

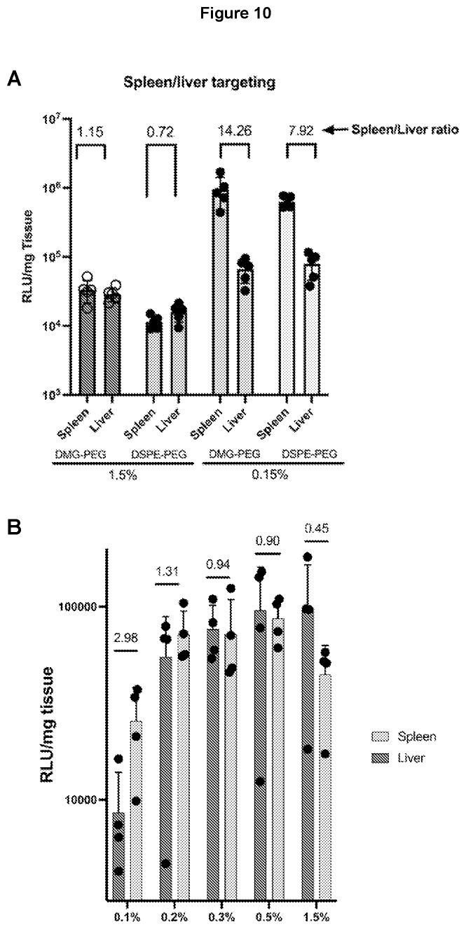

After IV injection the nanoluciferase levels in spleen and lymph nodes were consistently 50-100 fold higher for 0.15 mole % formulations than 1.5 mole % formulations. After IM injection the trend towards lymphoid tissues was also consistent but at 5-10 fold differences (FIG. 8). In particular, the IM injected 0.15 mole % DMG-PEG formulation produced significantly higher levels of nanoluciferase in the spleen and lower levels in the liver compared to the 1.5 mole % DMG-PEG formulation (FIG. 9), Both DMG-PEG and DSPE-PEG formulations containing 0.15 mole % PEGylated lipid rerouted the nanoluciferase mRNA out of the liver to the spleen (FIG. 10A).

### Example 7—PEGylated Lipid Content in Lipid Nanoparticles

Having established that PEGylated lipid content lower than 1.5 mole % produce a beneficial effect on LNP mRNA delivery, the inventors sought to determine the optimal range of PEGylated lipid for LNP formulations.

Varying DMG-PEG lipid compositions were tested ranging from 0.1 mole % to 1.5 mole %, and the cholesterol composition was accordingly adjusted. Nanoluciferase mRNA was packaged into the LNP formulations and the compositions were injected IM into BalbC mice (8-24 weeks).

TABLE 2
Tissue targeting to spleen after intramuscular injection
comparing 1.5 mole % and various lower % DMG-PEG formulations.
Percentage = mole % DMG-PEG content.

| Percentage | Ratio Spleen/Liver |
|---|---|
| 0.10% | 2.982 |
| 0.20% | 1.313 |
| 0.30% | 0.947 |
| 0.50% | 0.908 |
| 1.50% | 0.453 |

0.2 mole % to 0.5 mole % DMG-PEG resulted in the high expression of nanoluciferase in the spleen compared the standard 1.5 mole % PEGylated lipid formulations (Table 2), suggesting enhanced delivery spleen with this specific range. However, 0.1 mole % to 0.5 mole % DMG-PEG LNP formulations all hade a significantly higher spleen/liver expression ratio than 1.5 mole % DMG-PEG LNP formulations. A similar effect was also seen in the non-draining lymph with expression of nanoluciferase highest with 0.2 mole % DMG-PEG (data not shown). There was not a significant difference in nanoluciferase expression in the draining lymph nodes, quadriceps receiving the injection and liver, although 0.1 to 0.2 mole % had a mean expression level lower than the higher ranges (data not shown).

To further elucidate the PEGylated lipid range, the inventors test 0.15 mole %, 0.2 mole %, 0.25 mole %, 0.3 mole % and 0.35 mole % DMG-PEG in LNP formulations. As above, nanoluciferase mRNA was packaged into the LNP formulations and the compositions were injected IM. Similar to the previous results, the highest level of expression of nanoluciferase in the spleen, non-draining lymph nodes and lymph nodes was seen with 0.15 mole % with a trend of decreasing expression within increasing DMG-PEG content (data not shown).

Decreasing DMG-PEG content was associated with increasing spleen nanoluciferase expression and decreasing liver nanoluciferase expression (data not shown). Similarly, there is a relationship between decreasing DMG-PEG and increase non-draining lymph delivery and expression. Furthermore, lower concentrations of DMG-PEG rerouted the nanoluciferase expression from the liver to the spleen (Table 3).

TABLE 3
Tissue targeting to spleen after intramuscular
injection with lower % DMG-PEG formulations.
Percentage = mole % DMG-PEG content.

| Percentage | Ratio Spleen/Liver |
|---|---|
| 0.15% | 2.01 |
| 0.20% | 0.947 |
| 0.25% | 1.112 |
| 0.3% | 0.524 |
| 0.35% | 0.364 |

Together these results demonstrate that reduced PEGylation of the LNP, as formulated in the MIPS-LNPs enhances the uptake of particles and gene expression within the spleen. The MIPS-LNP formulation (using DODAP as an example) enhances the immune response which will likely increase efficiency both for prophylactic vaccines and therapeutic vaccines for cancer. Splenocytes targeting is attractive for many other applications such as expression of proteins for immunocheckpoint inhibition and other applications, through the targeted mRNA delivery by MIPS formulation, that induce antigen-specific tolerance, induction general tolerance and applications in countering autoimmune diseases, reducing inflammation driven by splenocytes or reducing allergy and anaphylactic reactions through means of mRNA delivery, siRNA delivery, DNA or any other nucleic acid or mRNA delivery with other molecules (Such as small drugs incorporated with MIPS LNPs). Conventional LNPs do not deliver to splenocytes and, therefore, are inferior when used for the applications outlined above.

It will be understood that the invention disclosed and defined in this specification extends to all alternative combinations of two or more of the individual features mentioned or evident from the text or drawings. All of these different combinations constitute various alternative aspects of the invention.

1. A lipid nanoparticle comprising:
(a) an active agent;
(b) a cationic and/or ionisable lipid comprising from about 40 mol % to about 60 mol % of the total lipid present in the nanoparticle;
(c) a phospholipid comprising from about 5 mol % to about 20 mol % of the total lipid present in the nanoparticle;
(d) a structural lipid comprising from about 30 mol % to about 50 mol % of the total lipid present in the nanoparticle;
(e) a PEGylated lipid comprising from about 0.05 mol % to less than 0.5 mol % of the total lipid present in the nanoparticle.

2. A lipid nanoparticle according to claim 1, wherein the PEGylated lipid comprises from about from about 0.06 mol % to about 0.5 mol %, from about 0.07 mol % to about 0.5 mol %, from about 0.08 mol % to about 0.5 mol %, from about 0.09 mol % to about 0.5 mol %, from about 0.1 mol % to about 0.5 mol %, from about 0.15 mol % to about 0.5 mol %, from about 0.2 mol % to about 0.5 mol %, from about 0.25 mol % to about 0.5 mol %, from about 0.3 mol % to about 0.5 mol %, from about 0.3 mol % to about 0.5 mol %, from about 0.35 mol % to about 0.5 mol %, from about 0.4 mol % to about 0.5 mol %, from about 0.45 mol % to about 0.5 mol %, from about 0.05 mol % to about 0.45 mol %, from about 0.05 mol % to about 0.4 mol %, from about 0.05 mol % to about 0.35 mol %, from about 0.05 mol % to about 0.3 mol %, from about 0.05 mol % to about 0.25 mol %, from about 0.05 mol % to about 0.2 mol %, from about 0.05 mol % to about 0.15 mol %, from about 0.05 mol % to about 0.1 mol %, from about 0.05 mol % to about 0.09 mol %, from about 0.05 mol % to about 0.08 mol %, from about 0.05 mol % to about 0.07 mol %, or from about 0.05 mol % to about 0.06 mol % of the total lipid present in the particle.

3. A lipid nanoparticle according to claim 1 or 2, wherein the PEGylated lipid comprises from 0.06 mol % to 0.5 mol %, from 0.07 mol % to 0.5 mol %, from 0.08 mol % to 0.5 mol %, from 0.09 mol % to 0.5 mol %, from 0.1 mol % to 0.5 mol %, from 0.15 mol % to 0.5 mol %, from 0.2 mol % to 0.5 mol %, from 0.25 mol % to 0.5 mol %, from 0.3 mol % to 0.5 mol %, from 0.3 mol % to 0.5 mol %, from 0.35 mol % to 0.5 mol %, from 0.4 mol % to 0.5 mol %, from 0.45 mol % to 0.5 mol %, from 0.05 mol % to 0.45 mol %, from 0.05 mol % to 0.4 mol %, from 0.05 mol % to 0.35 mol %, from 0.05 mol % to 0.3 mol %, from 0.05 mol % to 0.25 mol %, from 0.05 mol % to 0.2 mol %, from 0.05 mol % to 0.15 mol %, from 0.05 mol % to 0.1 mol %, from 0.05 mol % to 0.09 mol %, from 0.05 mol % to 0.08 mol %, from 0.05 mol % to 0.07 mol %, or from 0.05 mol % to 0.06 mol % of the total lipid present in the particle.

4. A lipid nanoparticle according to any one of claims 1 to 3, wherein the PEGylated lipid comprises 0.05 mol %, 0.06 mol %, 0.07 mol %, 0.08 mol %, 0.09 mol %, 0.1 mol %, 0.15 mol %, 0.2 mol %, 0.25 mol %, 0.3 mol %, 0.35 mol %, 0.4 mol %, or 0.45 mol % of the total lipid present in the particle.

5. A lipid nanoparticle according to any one of claims 1 to 4, wherein the PEGylated lipid is selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof.

6. A lipid nanoparticle according to any one of claims 1 to 4, wherein the PEGylated lipid is selected from the group consisting of PEG-c-DOMG, PEG-DMG, PEG-DLPE, PEG-DMPE, PEG-DPPC, or a PEG-DSPE lipid.

7. A lipid nanoparticle according to claim 6, wherein the PEGylated lipid is PEG-DSPE.

8. A lipid nanoparticle according to any one of claims 1 to 7, wherein the PEGylated lipid has a PEG component that has a molecular weight between about 100 Da and about 100,000 Da between about 100 Da and about 100,000 Da, between about 1000 Da and 9,000 Da, between about 1000 Da and 8,000 Da, between about 1000 Da and 7,000 Da, between about 1000 Da and 6,000 Da, between about 1000 Da and 5,000 Da, between about 1000 Da and 4,000 Da, between about 1000 Da and 3,000 Da, or between about 1000 Da and 2,000 Da.

9. A lipid nanoparticle according to any one of claims 1 to 7, wherein the PEGylated lipid has a PEG component that has a molecular weight of between about 1,000 Da and 5,000 Da, preferably between about 2,000 Da and 5,000 Da.

10. A lipid nanoparticle according to any one of claims 1 to 6, wherein the PEGylated lipid has a PEG component that has a molecular weight of 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da.

11. A lipid nanoparticle according to any one of claims 1 to 10, wherein the PEGylated lipid is DSPE-PEG, wherein the PEG has a molecular weight of 2000 Da.

12. A lipid nanoparticle according to any one of claims 1 to 11, wherein the cationic lipid is any one of N,N-dioleyl-N,N-dimethylammonium chloride (DODAC), 1,2-dioeoyloxy-3-(dimethylamino)propane (DODAP), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3.beta.-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 4-Hydroxybutyl)azanediyl)bis(hexane-6,1-diyl) bis(2-hexyldecanoate) (ALC-3015), 8-[(2-hydroxyethyl)[6-oxo-6-(undecyloxy)hexyl]amino]-octanoic acid,1-octylnonyl ester (SM-102) and mixtures thereof.

13. A lipid nanoparticle according to any one of claims 1 to 11, wherein the cationic lipid is of Formula I 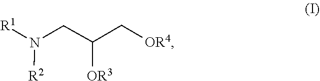
wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation, preferably the cationic lipid of Formula I is 1,2-dilinoleyloxy-N,N-dimethylaminopropane (DLinDMA) or 1,2-dilinolenyloxy-N,N-dimethylaminopropane (DLenDMA).

14. A lipid nanoparticle according to any one of claims 1 to 11, wherein the cationic lipid is of Formula II 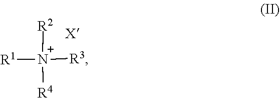
wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation.

15. A lipid nanoparticle according to any one of claims 1 to 11, wherein the cationic lipid is of Formula III 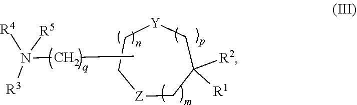
wherein R¹ and R² are either the same or different and independently optionally substituted C₁₂-C₂₄ alkyl, optionally substituted C₁₂-C₂₄ alkenyl, optionally substituted C₁₂-C₂₄ alkynyl, or optionally substituted C₁₂-C₂₄ acyl; R³ and R⁴ are either the same or different and independently optionally substituted C₁-C₆ alkyl, optionally substituted C₁-C₆ alkenyl, or optionally substituted C₁-C₅ alkynyl or R³ and R⁴ may join to form an optionally substituted heterocyclic ring of 4 to 6 carbon atoms and 1 or 2 heteroatoms chosen from nitrogen and oxygen; R⁵ is either absent or hydrogen or C₁-C₆ alkyl to provide a quaternary amine; m, n, and p are either the same or different and independently either 0 or 1 with the proviso that m, n, and p are not simultaneously 0; q is 0, 1, 2, 3, or 4; and Y and Z are either the same or different and independently O, S, or NH.

16. A lipid nanoparticle according to claim 15, wherein the cationic lipid of Formula III is 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), or mixtures thereof.

17. A lipid nanoparticle according to any one of claims 1 to 16, wherein the cationic lipid is DODAP, DLin-DMA, DLin-K-DMA, DLin-K2-DMA DLin-MC3-DMA.

18. A lipid nanoparticle according to any one of claims 1 to 17, wherein the cationic lipid comprises from about 40 mol % to about 60 mol %, from about 40 mol % to about 55 mol %, from about 40 mol % to about 50 mol %, from about 40 mol % to about 45 mol %, from about 45 mol % to about 60 mol %, from about 50 mol % to about 60 mol %, or from about 55 mol % to about 60 mol % of the total lipid present in the particle.

19. A lipid nanoparticle according to any one of claims 1 to 18, wherein the cationic lipid comprises about 40, 41, 42, 43, 44, 45, 46, 47, 48, 49, 50, 51, 52, 53, 54, 55, 56, 57, 58, 59 or 60 mol % of the total lipid present in the particle.

20. A lipid nanoparticle according to any one of claims 1 to 19, wherein the phospholipid is a cationic phospholipid

21. A lipid nanoparticle according to any one of claims 1 to 20, wherein the phospholipid is an unsaturated lipid.

22. A lipid nanoparticle according to any one of claims 1 to 20, wherein the phospholipid is a polyunsaturated lipid.

23. A lipid nanoparticle according to any one of claims 1 to 22, wherein the phospholipid is according to Formula (IV): 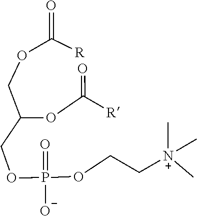
in which represents a phospholipid moiety and R and R, represent fatty acid moieties with or without unsaturation that may be the same or different.

24. A lipid nanoparticle according to any one of claims 1 to 23, wherein the phospholipid moiety is selected from the group consisting of: phosphatidyl choline, phosphatidyl ethanolamine, phosphatidyl glycerol, phosphatidyl serine, phosphatidic acid, 2-lysophosphatidyl choline, and a sphingomyelin.

25. A lipid nanoparticle according to any one of claims 1 to 24, wherein the phospholipid has a fatty acid moiety selected from the non-limiting group consisting of: lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

26. A lipid nanoparticle according to any one of claims 1 to 23, wherein the phosopholipid is lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof.

27. A lipid nanoparticle according to any one of claims 1 to 26, wherein the phosopholipid is distearoylphosphatidylcholine (DSPC).

28. A lipid nanoparticle according to any one of claims 1 to 27, wherein the phospholipid comprises from about 5 mol % to about 20 mol %, from about 5 mol % to about 15 mol %, from about 5 mol % to about 10 mol %, from about 10 mol % to about mol %, or from about 15 mol % to about 20 mol % of the total lipid present in the particle.

29. A lipid nanoparticle according to any one of claims 1 to 27, wherein the phospholipid typically comprises from 5 mol % to 20 mol %, from 5 mol % to 15 mol %, from 5 mol % to 10 mol %, from 10 mol % to 20 mol %, or from 15 mol % to 20 mol % of the total lipid present in the particle.

30. A lipid nanoparticle according to any one of claims 1 to 29, wherein the. structural lipid is selected from the group consisting of cholesterol, fecosterol, sitosterol, ergosterol, campesterol, stigmasterol, brassicasterol, tomatidine, tomatine, ursolic acid, alpha-tocopherol, and mixtures thereof.

31. A lipid nanoparticle according to any one of claims 1 to 30, wherein the structural lipid is cholesterol.

32. A lipid nanoparticle according to any one of claims 1 to 31, wherein the structural lipid comprises from about 30 mol % to about 50 mol %, from about 30 mol % to about 45 mol %, from about 30 mol % to about 40 mol %, from about 30 mol % to about 35 mol %, from about 35 mol % to about 50 mol %, from about 40 mol % to about 50 mol %, or from about 45 mol % to about 50 mol % of the total lipid present in the particle.

33. A lipid nanoparticle according to any one of claims 1 to 31, wherein the structural lipid typically comprises from 30 mol % to 50 mol %, from 30 mol % to 45 mol %, from 30 mol % to 40 mol %, from 30 mol % to 35 mol %, from 35 mol % to 50 mol %, from 40 mol % to 50 mol %, or from 45 mol % to 50 mol % of the total lipid present in the particle.

34. A lipid nanoparticle according to any one of claims 1 to 33, wherein the active agent comprises or consists of a peptide or polypeptide.

35. A lipid nanoparticle according to claim 34, wherein the polypeptide comprises an antibody, preferably a polyclonal antibody, a monoclonal antibody, an antibody fragment; a humanized antibody, a recombinant antibody, a recombinant human antibody, a Primatized™ antibody, or mixtures thereof.

36. A lipid nanoparticle according to claim 34, wherein the peptide or polypeptide comprises a cytokine, a growth factor, an apoptotic factor, a differentiation-inducing factor, a cell-surface receptor, a ligand, a hormone, a small molecule (e.g., small organic molecule or compound), or mixtures thereof.

37. A lipid nanoparticle according to any one of claims 1 to 33, wherein the active agent comprises or consists of a nucleic acid.

38. A lipid nanoparticle according to claim 37, wherein the nucleic acid comprises an interfering RNA molecule, preferably an siRNA, aiRNA, miRNA, or mixtures thereof.

39. A lipid nanoparticle according to claim 37, wherein the nucleic acid comprises single-stranded or double-stranded DNA, RNA, or a DNA/RNA hybrid, preferably an antisense oligonucleotide, a ribozyme, a plasmid, an immunostimulatory oligonucleotide, or mixtures thereof.

40. A lipid nanoparticle according to any one of claims 1 to 39, wherein the active agent is mRNA.

41. A lipid nanoparticle according to any one of claims 1 to 40, the active agent is fully encapsulated within the lipid portion of the lipid particle such that the active agent or therapeutic agent in the lipid particle is resistant in aqueous solution to enzymatic degradation, e.g., by a nuclease or protease.

42. A lipid nanoparticle according to any one of claims 1 to 41, wherein the lipid nanoparticle does not comprise a targeting ligand that specifically binds to a molecule on the surface of the target cell.

43. A lipid nanoparticle according to any one of claims 1 to 342, wherein the mean size of the lipid nanoparticle is greater than about 100 nm e.g., measured by dynamic light scattering (DLS).

44. A lipid nanoparticle according to any one of claims 1 to 43, wherein the mean size may be greater than, or great than about, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, or 500 nm.

45. A lipid nanoparticle according to any one of claims 1 to 43, wherein the mean size of a nanoparticle of the invention may be from about 75 nm to about 500 nm, from about 80 nm to about 500 nm, from about 90 nm to about 500 nm, from about 100 nm to about 500 nm, from about 110 nm to about 500 nm, from about 120 nm to about 500 nm, from about 130 nm to about 500 nm, from about 140 nm to about 500 nm, from about 150 nm to about 500 nm, from about 160 nm to about 500 nm, from about 170 nm to about 500 nm, from about 180 nm to about 500 nm, from about 190 nm to about 500 nm, from about 200 nm to about 500 nm, from about 210 nm to about 500 nm, from about 220 nm to about 500 nm, from about 230 nm to about 500 nm, from about 240 nm to about 500 nm, from about 250 nm to about 500 nm, from about 260 nm to about 500 nm, from about 270 nm to about 500 nm, from about 280 nm to about 500 nm, from about 300 nm to about 500 nm, from about 310 nm to about 500 nm, from about 320 nm to about 500 nm, from about 330 nm to about 500 nm, from about 340 nm to about 500 nm, from about 350 nm to about 500 nm, from about 360 nm to about 500 nm, from about 370 nm to about 500 nm, from about 380 nm to about 500 nm, from about 390 nm to about 500 nm, from about 400 nm to about 500 nm, from about 410 nm to about 500 nm, from about 420 nm to about 500 nm, from about 430 nm to about 500 nm, from about 440 nm to about 500 nm, from about 450 nm to about 500 nm, from about 460 nm to about 500 nm, from about 470 nm to about 500 nm, from about 480 nm to about 500 nm, or from about 490 nm to about 500 nm.

46. A lipid nanoparticle according to any one of claims 1 to 45, wherein the mean size of a nanoparticle of the invention may be from about 100 nm to about 490 nm, from about 100 nm to about 480 nm, from about 100 nm to about 470 nm, from about 100 nm to about 460 nm, from about 100 nm to about 450 nm, from about 100 nm to about 440 nm, from about 100 nm to about 430 nm, from about 100 nm to about 420 nm, from about 100 nm to about 430 nm, from about 100 nm to about 420 nm, from about 100 nm to about 410 nm, from about 100 nm to about 400 nm, from about 100 nm to about 390 nm, from about 100 nm to about 380 nm, from about 100 nm to about 370 nm, from about 100 nm to about 360 nm, from about 100 nm to about 350 nm, from about 1000 nm to about 340 nm, from about 100 nm to about 330 nm, from about 100 nm to about 320 nm, from about 100 nm to about 310 nm, from about 100 nm to about 300 nm, from about 100 nm to about 290 nm, from about 100 nm to about 280 nm, from about 100 nm to about 270 nm, from about 100 nm to about 260 nm, from about 100 nm to about 250 nm, from about 100 nm to about 240 nm, from about 100 nm to about 230 nm, from about 100 nm to about 220 nm, from about 100 nm to about 210 nm, from about 100 nm to about 200 nm, from about 100 nm to about 190 nm, from about 100 nm to about 180 nm, from about 100 nm to about 170 nm, from about 100 nm to about 160 nm, from about 100 nm to about 150 nm, from about 100 nm to about 140 nm, from about 100 nm to about 130 nm, from about 100 nm to about 120 nm, or from about 100 nm to about 110 nm.

47. A lipid nanoparticle according to any one of claims 1 to 46, wherein the lipid nanoparticle has a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

48. A lipid nanoparticle according to any one of claims 1 to 46, wherein the lipid nanoparticle has a polydispersity index of about 0.10 to about 0.20.

49. A lipid nanoparticle according to any one of claims 1 to 48, wherein the zeta potential of a nanoparticle composition may be from about −50 mV to about +10 mV, preferably about −20 mV to about +5 mV.

50. A pharmaceutical composition comprising a lipid nanoparticle according to any one of claims 1 to 49, and a pharmaceutically acceptable carrier, diluent or excipient.

51. A method for introducing an active agent into a cell, preferably the cell is present in vivo, the method comprising contacting the cell with a lipid nanoparticle according to any one of claims 1 to 49, thereby introducing the active agent (e.g. nucleic acid) into the cell.

52. A method for the in vivo delivery of an active agent, the method comprising administering to a subject in need thereof a lipid nanoparticle according to any one of claims 1 to 49, or a pharmaceutical composition according to claim 50, thereby delivering the active agent in vivo.

53. A method for treating or preventing a disease or condition in a subject in need thereof, the method comprising administering to the subject a lipid nanoparticle according to any one of claims 1 to 49 or pharmaceutical composition according to claim 50, thereby treating or preventing a disease or condition in a subject in need thereof.

54. A method of producing a polypeptide of interest in a cell, preferably a mammalian cell, the method comprising contacting the cell with a lipid nanoparticle according to any one of claims 1 to 47, or a pharmaceutical composition according to claim 50, wherein the active agent is an mRNA encoding the polypeptide of interest, wherein the mRNA is capable of being translated in the cell to produce the polypeptide of interest.

55. A method of delivering an mRNA into a cell, preferably a mammalian cell, the method comprising administering to a subject a lipid nanoparticle according to any one of claims 1 to 49, or a pharmaceutical composition according to claim 50, wherein the active agent is an mRNA, thereby delivering an mRNA into a cell.

56. A method according to any one of claims 51, 54 or 55, wherein the cell is a cell located in the spleen.

57. A method according to claim 56, wherein the cell located in the spleen is a cell of the spleen or a cell originating in another part of the subject that is trafficked to the spleen.

58. A method for the delivery of an mRNA to a target tissue, the method comprising administering to a subject a lipid nanoparticle according to any one of claims 1 to 49, or a pharmaceutical composition according to claim 50, wherein the active agent is an mRNA, thereby delivering the mRNA to a target tissue.

59. A method of claim 55, wherein the target tissue is a mammalian spleen.

60. A lipid nanoparticle according to any one of claims 1 to 49, or a pharmaceutical composition according to claim 50 in the manufacture of a medicament for treating or preventing a disease or condition in a subject in need thereof.

61. A lipid nanoparticle according to any one of claims 1 to 49, or a pharmaceutical composition according to claim 50 for use in the treatment or prevention of a disease or condition in a subject in need thereof.
